(19) United States
(12) Patent Application Publication
(10) Pub. No.: US 2003/0220383 A1
(43) Pub. Date: Nov. 27, 2003

(54) Use of inhibitors of the sodium-dependent chloride/bicarbonate exchanger for the treatment of thrombotic and inflammatory disorders

(75) Inventors: Hans-Jochen Lang, Hofheim (DE); Heinz-Werner Kleemann, Bischofsheim (DE); Stefan Werner Schneider, Muenster (DE); Hans Oberleithner, Muenster (DE); Andre Niemeyer, Muenster (DE)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: AVENTIS PHARMA DEUTSCHLAND GMBH, Frankfurt (DE)

(21) Appl. No.: 10/367,546

(22) Filed: Feb. 14, 2003

Related U.S. Application Data
(60) Provisional application No. 60/366,756, filed on Mar. 22, 2002.

(30) Foreign Application Priority Data
Feb. 14, 2002 (DE) 10206354.0

(51) Int. Cl. A61K 31/4162; A61K 31/18

(52) U.S. Cl. 514/396; 514/397; 514/603

(57) ABSTRACT

Inhibitors of the cellular sodium-dependent chloride/bicarbonate exchangers show an inhibiting effect on the secretion of von-Willebrand factor. These inhibitors can therefore be employed for the treatment of thrombotic and inflammatory disorders.

### FIELD OF THE INVENTION

The invention relates to the use of inhibitors of the sodium-dependent chloride/bicarbonate exchanger in human and veterinary medicine for the prevention and treatment of acute or chronic diseases caused by elevated levels of von Willebrand factor in the blood. The inhibitors can therefore be employed for the treatment of thrombotic and inflammatory disorders.

It has now been found that the compounds employed according to the invention inhibit the release of von Willebrand factor from endothelial cells. The compounds of the invention inhibit the massive pH-dependent release of vWF that accumulates during ischemia. It has now been found that there is a delay and reduction in the release of vWF as the pH falls. It has also been found that the compounds of the invention inhibit increased P-selectin expression in the endothelial cell membrane.

### BACKGROUND OF THE INVENTION

Inhibitors of the cellular sodium-dependent chloride/bicarbonate exchanger (NCBE) are known (EP 0 855 392, EP 1 097 140). NCBE inhibitors are suitable because of the inhibition of the cellular Na⁺-dependent Cl⁻/HCO₃⁻ exchange mechanism for the prevention and treatment of arrhythmias, of infarctions and of angina pectoris and generally for cardioprotection, especially following ischemia and reperfusion events. In addition, these NCBE inhibitors can be used, because of their potentially protective effects in pathological hypoxic and ischemic situations, for the treatment of all acute or chronic damage caused by ischemia or diseases induced primarily or secondarily thereby. Further potential indications for possible use are protection of tissues for organ transplants, renal failure, protection from ischemia-induced damage of the central nervous system, treatment of states of shock, diseases induced by proliferation, including cancer, fibrotic disorders, organ hypertrophies and hyperplasias, impairments of respiratory drive etc. However, no use of NCBE inhibitors for the prevention of thrombus formation per se is described.

The mechanism of action of NCBE inhibitors that operates in the acute ischemic event comprises their reduction of the enhanced influx of sodium ions that arises in acutely hypoperfused tissue due to activation of the sodium/hydrogen exchanger (NHE) as a consequence of intracellular acidification. This delays the situation of tissue sodium overload. Since there is coupling of sodium and calcium ion transport in cardiac tissue, this prevents the life-threatening calcium overload of heart cells. In addition, with NCBE inhibitors there is suppression or delay of the realkalinization of the interior of cells owing to blockage of bicarbonate influx.

Thus, of course, as expected no protective effects of NCBE inhibitors against these acute events were observable where blood flow was normal and healthy.

Numerous classes of substances that intervene in the interplay of coagulation factors and thus cause cessation of the coagulation cascade are described in the prior art. Likewise, numerous action principles that do not suppress thrombus formation, but cause the dissolution (lysis) of thrombi that have already formed, have been developed. Some of these action principles, which intervene at a wide variety of junction points in said cascade, have been introduced into therapy to prevent thrombogenesis, such as derivatives of the vitamin K group (phylloquinones), factor VIII and factor IX products, platelet aggregation inhibitors such as acetylsalicylic acid, dipyridamole and ticlopidine, anticoagulants such as heparins or heparinoids.

The blood coagulation cascade can be divided mechanistically into two pathways as depicted in the following diagram,

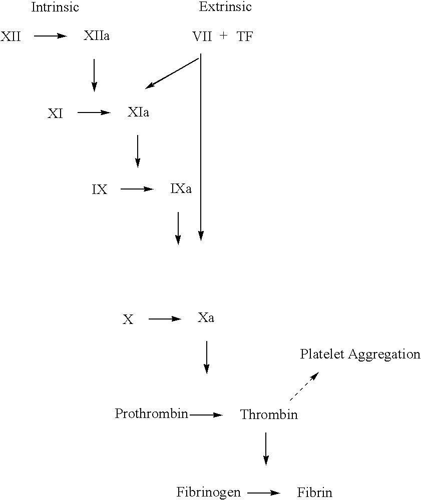

namely into an intrinsic and an extrinsic route, the two of which finally meet in the activation of factor X and the resulting generation of thrombin and subsequently of fibrin.

It is important in the therapeutic use of such blood coagulation inhibitors that the inhibition of coagulation achieved is not too strong or complete, which would inhibit the formation of microthrombi and microcoagulations that are vital and that must take place at the microtraumata that are continually happening. Only imprecise adjustment of the degree of inhibition of coagulation is possible as a result of differences in the response of the particular individual at a particular time, and the degree must be carefully monitored where possible. If these many small coagulation processes that are permanently taking place are inhibited there is a high risk of extensive hemorrhage (hemophilia).

The disadvantage of the known therapeutic agents available on the market that intervene as inhibitors in the coagulation event is therefore the high risk of bleeding complications. The risk of life-threatening hemorrhage exists especially during high-dose thrombolysis therapy, e.g., during therapy of acute myocardial infarction or pulmonary embolism. Thus, there is an urgent need for therapeutic agents that do not entail a risk of increased tendency to bleeding despite overdosage.

Many of the known anticoagulant substances act by exerting an effect on the blood platelets, the thrombocytes, and inhibiting their function or inhibiting their activation. The endothelium also evidently plays a central part in the coagulation event. Thus, for example, the von Willebrand factor (vWF) that is necessary for coagulation is produced for the most part in endothelial cells and is secreted by them permanently (constitutively) into the circulating blood in order to ensure the necessary coagulation processes in the blood. A-considerable part of the produced vWF is stored in cytoplasmic granules, called Weibel-Palade bodies, and released as required through stimulation of endothelial cells. If endothelial cells are unable to produce vWF and deliver it to the blood, the result is the well known genetic vWF-dependent disease, von Willebrand-Jurgens syndrome, which is characterized by hemorrhages that can scarcely be stopped. It is only in recent years that disorders caused by elevated concentrations of vWF in the blood, thus inducing, for example, an increased tendency to blood coagulation and inflammatory processes, have become known. Thus, Kamphuisen et al. demonstrate on the basis of a large number of studies in their publication “Elevated factor VIII levels and the risk of thrombosis” (Arterioscler. Thromb. Vasc. Biol. 21(5):731-738 (2001)) that there is a significant association between elevated vWF levels in the blood and an increased rate of thrombotic disorders. Factor VIII forms with vWF a complex as necessary precondition for blood coagulation. It has been possible to establish that high levels of von Willebrand factor and (vWF) and of vWF-bound factor VIII in the blood represent a clear thrombosis risk factor. However, antithrombotic agents that antagonize the stabilizing binding of vWF to factor VIII may also be disadvantageous because, in the event of overdosage, substantial inhibition of blood coagulation and dangerous tendencies to bleeding must be expected.

Thus, it would be useful to have effective compounds for the treatment of acute or chronic diseases caused by elevated levels of von Willebrand factor in the blood which secretion takes place normally and constitutively at the normal pH of blood that is known to be about 7.4. Part of the vWF is stored in Weibel-Palade bodies. Exocytosis of the Weibel-Palade bodies in that the vWF is packaged is increasingly inhibited as the pH declines. Thus, under acidotic conditions, there is a significant increase in Weibel-Palade bodies and thus extensive accumulation of vWF in the endothelial cell, and a reduced constitutive and stimulated vWF secretion. This can be visualized by staining procedures and demonstrated by quantitative measurements of vWF in the supernatant. Such acidotic states with significant pH reductions below 7 occur, for example, in cases of tissue ischemia. At the instant of realkalinization and endothelial cell stimulation, which corresponds to the reperfusion state, within seconds exocytosis takes place, and thus emptying of the Weibel-Palade bodies (WPB), thus leading to massive release of the prothrombotic risk factor.

Furthermore, besides vWF, the Weibel-Palade bodies also store the transmembrane protein P-selectin (Wagner, D. D. 1993, Thromb. Haemost., 70:105-110). P-Selectin is located in the vesicle membrane and, after vesicle fusion (exocytosis), is incorporated into the plasma membrane of the endothelial cell. This means that every Weibel-Palade body exocytosis leads not only to increased vWF release but also to increased P-selectin expression in the endothelial cell membrane. The examples show vWF secretion (quantitive measurement by ELISA) during acidosis and during subsequent reperfusion. In parallel, these quantitative measurements are confirmed by immunofluorescence data on the Weibel-Palade bodies. The measured vWF is thus not only a marker of increased (increase in vWF secretion) or reduced (decrease in vWF secretion) tendency to thrombosis (via increase in platelet aggregation), but also a direct marker of increased or reduced P-selectin expression in the endothelial cell membrane. P-Selectin serves as anchor for leukocytes and thus the initial inflammatory reaction (Vestweber, D., Blanks, J. E. 1999, Physiol. Rev., 79:181-213; Issekutz, A. C., Issekutz, T. B. 2002, J. Immunol., 168:1934-1939). The pathophysiological significance is wide-ranging and confirmed for ischemia/reperfusion disorders, thromboses and arteriosclerosis (Massberg, S., et al., 1998, Blood, 92:507-515; Kita, T., et al., 2001, Ann. N.Y. Acad. Sci., 947:199-205). Besides the significance of P-selectin as marker of inflammation and initiator of inflammation, it plays an essential part in the process of cancer dissemination (Varki, A., Varki, N. M. 2001, Braz. J. Med. Biol. Res. 34:711-717) and during various inflammations of joints (arthritis) (Veihelmann, A. et al, 1999, Microcirculation, 6: 281-290; McInnes, I. B., et al., 2001, J. Immunol., 167:4075-4082). Thus the mode of action of the substances described here may also find use as therapeutic agent for all the abovementioned P-selectin-associated disorders.

### SUMMARY OF THE INVENTION

The invention relates to a method for the prophylactic or therapeutic treatment of a patient that would be subject to an acute or chronic disease caused by elevated levels of von Willebrand factors in the blood or increased expression of P-selectin, comprising administering a therapeutically effective amount of a cellular sodium-dependent chloride/bicarbonate exchanger inhibitor to the patient.

### DETAILED DESCRIPTION OF THE INVENTION

Definitions

Alkyl radicals and alkylene radicals may be straight-chain or branched. This also applies to the alkylene radicals of the formulae CₐH₂ₐ,C_{b}H_{2b},C_{d}H_{2d},C_{g}H_{2g}, and CₗH₂ₗ. Alkyl radicals and alkylene radicals may also be straight-chain or branched when they are substituted or present in other radicals, e.g., in an alkoxy radical or in an alkylmercapto radical or in a fluorinated alkyl radical.

Examples of alkyl radicals having 1, 2, 3, 4, 5, 6, 7 or 8 carbon atoms are: methyl, ethyl, n-propyl, n-butyl, pentyl, hexyl, heptyl, octyl, isopropyl, isobutyl, isopentyl, neopentyl, isohexyl, 3-methylpentyl, sec-butyl, tert-butyl, tert-pentyl. The divalent radicals derived from these radicals, e.g., methylene, 1,1-ethylene, 1,2-ethylene, 1,1-propylene, 1,2-propylene, 2,2-propylene, 1,3-propylene, 1,4-butylene, 1,5-pentylene, 2,2-dimethyl-1,3-propylene, 1,6-hexylene, etc. are examples of alkylene radicals.

Cycloalkyl means a carbocyclic ring radicals having 3, 4, 5, 6 or 7 carbon atoms, in particular, cyclopropyl, cyclobutyl, cyclopentyl, cyclohexyl, cycloheptyl which may, however, also be substituted by alkyl having 1, 2, 3 or 4 carbon atoms. 4-Methylcyclohexyl and 2,3-dimethylcyclopentyl may be mentioned as examples of substituted cycloalkyl radicals.

Heterocylyl means a carbocyclic ring radicals having 3, 4, 5, or 6 carbon atoms, wherein one or more ring carbon atoms is replaced by a heteroatom selected from N, O or S. For example piperdinyl, pyrrolyl, tetrahydrofuranyl, or tetrahydrothiophenyl.

Heteroaryl having 1, 2, 3, 4, 5, 6, 7, 8 or 9 carbon atoms means, in particular, radicals derived from phenyl or naphthyl in which one or more CH groups are replaced by N and/or in which at least two adjacent CH groups are replaced by S, NH or O (to form a five-membered aromatic ring). It is further possible for one or both atoms at the fusion site in bicyclic radicals (as in indolizinyl) to be nitrogen atoms.

Heteroaryl is, in particular, furanyl, thienyl, pyrrolyl, imidazolyl, pyrazolyl, triazolyl, tetrazolyl, oxazolyl, isoxazolyl, thiazolyl, isothiazolyl, pyridyl, pyrazinyl, pyrimidinyl, pyridazinyl, indolyl, indazolyl, quinolyl, isoquinolyl, phthalazinyl, quinoxalinyl, quinazolinyl, cinnolinyl. Nitrogen-containing heterocycles having 1, 2, 3, 4, 5, 6, 7, 8 or 9 carbon atoms are, in particular, the aromatic systems 1-, 2- or 3-pyrrolyl, 1-, 2-, 4- or 5-imidazolyl, 1-, 3-, 4- or 5-pyrazolyl, 1,2,3-triazol-1-, -4- or 5-yl, 1,2,4-triazol-1-, -3- or -5-yl, 1- or 5-tetrazolyl, 2-, 4- or 5-oxazolyl, 3-, 4- or 5-isoxazolyl, 1,2,3-oxadiazol-4- or 5-yl, 1,2,4-oxadiazol-3-or 5-yl, 1,3,4-oxadiazol-2-yl or -5-yl, 2-, 4- or 5-thiazolyl, 3-, 4- or 5-isothiazolyl, 1,3,4-thiadiazol-2- or -5-yl, 1,2,4-thiadiazol-3- or -5-yl, 1,2,3-thiadiazol-4- or 5-yl, 2-, 3- or 4-pyridyl, 2-, 4-, 5- or 6-pyrimidinyl, 3- or 4-pyridazinyl, pyrazinyl, 1-, 2-, 3-, 4-, 5-, 6- or 7-indolyl, 1-, 2-, 4- or 5-benzimidazolyl, 1-, 3-, 4-, 5-, 6- or 7-indazolyl, 2-, 3-, 4-, 5-, 6-, 7- or 8-quinolyl, 1-, 3-, 4-, 5-, 6-, 7- or 8-isoquinolyl, 2-, 4-, 5-, 6-, 7- or 8-quinazolinyl, 3-, 4-, 5-, 6-, 7- or 8-cinnolinyl, 2-, 3-, 5-, 6-, 7- or 8-quinoxalinyl, 1-, 4-, 5-, 6-, 7- or 8-phthalazinyl.

The nitrogen-containing heterocycles are particularly preferably pyrrolyl, imidazolyl, quinolyl, pyrazolyl, pyridyl, pyrazinyl, pyrimidinyl and pyridazinyl.

Thienyl stands for both 2- and for 3-thienyl. furyl stands for 2- and 3-furyl. Monosubstituted phenyl radicals may be substituted in position 2, 3 or 4, disubstituted in position 2,3, 2,4, 2,5, 2,6, 3, 4 or 3,5, trisubstituted in position 2,3,4, 2,3,5, 2,3,6, 2,4,5, 2,4,6 or 3,4,5. Corresponding statements also apply analogously to the nitrogen-containing heterocycles or the thiophene radical.

If the moiety is di- or trisubstituted, the substituents may be identical or different.

Particular Embodiments

The invention further relates to the method wherein the cellular sodium-dependent chloride/bicarbonate exchanger inhibitor is a compound of the formula I

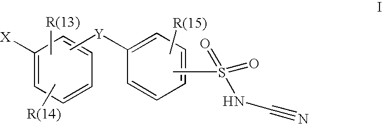

or a stereoisomeric form thereof or a mixture of stereoisomeric forms in any ratio, or a physiologically tolerated salt thereof, wherein:

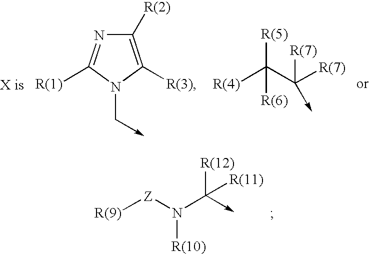

R(1) is hydrogen, alkyl having 1, 2, 3, 4, 5, 6, 7 or 8 carbon atoms, —CₐH₂ₐ-phenyl, which phenyl is unsubstituted or substituted by 1, 2 or 3 identical or different substituents selected from F, Cl, Br, I, CF₃, methyl, methoxy, hydroxyl and —NR(19)R(20), —C_{b}H_{2b}-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(21)R(22), or —C_{c}H_{2c}-cycloalkyl, which cycloalkyl has 3, 4, 5, 6 or 7 carbon atoms;

R(2) and R(3) are independently hydrogen, F, Cl, Br, I, CF₃, —CN, —NO₂, —CH₂OR(23), —CO—R(24), —O—R(25), —O-(alkylenyl having 2, 3, or 4 carbon atoms)-O—R(23), —NR(92)R(93), alkyl having 1, 2, 3, 4, 5, 6, 7 or 8 carbon atoms, cycloalkyl having 3, 4, 5, 6 or 7 carbon atoms, —C_{d}H_{2d}-phenyl, which phenyl is unsubstituted or substituted by 1, 2 or 3 identical or different substituents selected from F, Cl, Br, I, CF₃, methyl, methoxy, hydroxyl and —NR(33)R(34), —CₑH₂ₑ-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(35)R(36), or —SO_{f}—R(37);

R(4) is hydrogen, alkyl having 1, 2, 3, 4, 5, 6, 7 or 8 carbon atoms, 1-naphthyl, 2-naphthyl, —CᵢH₂ᵢ-cycloalkyl, which cycloalkyl has 3, 4, 5, 6 or 7 carbon atoms, or —CᵢH₂ᵢ-phenyl, which phenyl moiety is unsubstituted or substituted by 1, 2 or 3 identical or different substituents selected from alkyl having 1, 2, 3, 4, 5, 6, 7 or 8 carbon atoms, F, Cl, Br, I, CF₃, —SOⱼR(48), —OR(49), —NR(50)R(51), —CN, —NO₂ or —CO—R(52), or R(4) and R(6) taken together with the carbon atom bearing them form cycloalkyl, which cycloalkyl has 3, 4, 5, 6 or 7 carbon atoms, or fluorenyl;

R(5), R(6), R(7) and R(8) are independently hydrogen, F, CF₃, —O—R(56), alkyl having 1, 2, 3, 4, 5, 6, 7 or 8 carbon atoms, cycloalkyl having 3, 4, 5, 6 or 7 carbon atoms, —CₖH₂ₖ-phenyl, which the phenyl moiety is unsubstituted or substituted by 1, 2 or 3 identical or different substituents selected from F, Cl, Br, I, CF₃, methyl, methoxy, hydroxyl and —NR(57)R(58), or R(5) and R(7) taken together form a second bond between the carbon atoms bearing them;

R(9) is alkyl having 1, 2, 3, 4, 5, 6, 7 or 8 carbon atoms, alkenyl having 2, 3, 4, 5, 6, 7 or 8 carbon atoms, or -Q(1)-A;

R(10) is hydrogen, alkyl having 1, 2, 3, 4, 5, 6, 7 or 8 carbon atoms; alkenyl having 2, 3, 4, 5, 6, 7 or 8 carbon atoms, or -Q(2)—B,

R(11) and R(12) are independently hydrogen or alkyl having 1, 2, 3, 4, 5, 6, 7 or 8 carbon atoms;

R(13), R(14) and R(15) are independently hydrogen, alkyl having 1, 2, 3, 4, 5, 6, 7 or 8 carbon atoms, F, Cl, Br, I, CF₃, —CN, —NO₂, —SO_{q}—R(79), —CO—R(80), —O—R(81) or —O-(alkylenyl having 2, 3 or 4 carbon atoms)-O—R(95);

R(16) is hydrogen or —OR(85);

R(17) is hydrogen or alkyl having 1, 2, 3, 4, 5, 6, 7 or 8 carbon atoms;

R(18) is hydrogen, alkyl having 1, 2, 3, 4, 5, 6, 7 or 8 carbon atoms, —CO—R(87) or —SO₂R(88);

R(19) and R(20) are independently hydrogen or alkyl having 1, 2, 3 or 4 carbon atoms;

R(21) and R(22) are independently hydrogen or alkyl having 1, 2, 3 or 4 carbon atoms;

R(23) is hydrogen or alkyl having 1, 2, 3, 4, 5, 6, 7 or 8 carbon atoms;

R(23) is hydrogen or alkyl having 1, 2, 3, 4, 5, 6, 7 or 8 carbon atoms;

R(24) is hydrogen, alkyl having 1, 2, 3, 4, 5, 6, 7 or 8 carbon atoms, —OR(26), or phenyl, which phenyl is unsubstituted or substituted by 1, 2 or 3 identical or different substituents selected from F, Cl, Br, I, CF₃, methyl, methoxy, hydroxyl and —NR(27)R(28);

R(25) is hydrogen, alkyl having 1, 2, 3, 4, 5, 6, 7 or 8 carbon atoms, phenyl, which phenyl is unsubstituted or substituted by 1, 2 or 3 identical or different substituents selected from F, Cl, Br, I, CF₃, methyl, methoxy, hydroxyl and —NR(29)R(30), or heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(31)R(32);

R(26) is hydrogen or alkyl having 1, 2, 3, 4, 5, 6, 7 or 8 carbon atoms;

R(27) and R(28) are independently hydrogen or alkyl having 1, 2, 3 or 4 carbon atoms;

R(29) and R(30) are independently hydrogen or alkyl having 1, 2, 3 or 4 carbon atoms;

R(31) and R(32) are independently hydrogen or alkyl having 1, 2, 3 or 4 carbon atoms;

R(33) and R(34) are independently hydrogen or alkyl having 1, 2, 3 or 4 carbon atoms;

R(35) and R(36) are independently hydrogen or alkyl having 1, 2, 3 or 4 carbon atoms;

R(37) is alkyl having 1, 2, 3, 4, 5, 6, 7 or 8 carbon atoms, cycloalkyl having 3, 4, 5, 6 or 7 carbon atoms, or —C_{g}H_{2g}-phenyl, which phenyl is unsubstituted or substituted by 1, 2 or 3 identical or different substituents selected from F, Cl, Br, I, CF₃, methyl, methoxy, hydroxyl and —NR(38)R(39);

R(38) and R(39) are independently hydrogen or alkyl having 1, 2, 3 or 4 carbon atoms;

R(48) is alkyl having 1, 2, 3 or 4 carbon atoms or —NR(53)R(54);

R(49) is hydrogen or alkyl having 1, 2, 3 or 4 carbon atoms;

R(50) and R(51) are independently hydrogen or alkyl having 1, 2, 3 or 4 carbon atoms;

R(52) is hydrogen, alkyl having 1, 2, 3, 4, 5, 6, 7 or 8 carbon atoms or —OR(55);

R(53) and R(54) are independently hydrogen or alkyl having 1, 2, 3 or 4 carbon atoms;

R(55) is hydrogen, alkyl having 1, 2, 3, 4, 5, 6, 7 or 8 carbon atoms;

R(56) is hydrogen, alkyl having 1, 2, 3, 4, 5, 6, 7 or 8 carbon atoms, or phenyl which is unsubstituted or substituted by 1, 2 or 3 identical or different substituents selected from F, Cl, Br, I, CF₃, methyl, methoxy, hydroxyl and —NR(59)R(60), or heteroaryl having 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(61)R(62);

R(57) and R(58) are independently hydrogen or alkyl having 1, 2, 3 or 4 carbon atoms;

R(59) and R(60) are independently hydrogen or alkyl having 1, 2, 3 or 4 carbon atoms;

R(61) and R(62) are independently hydrogen or alkyl having 1, 2, 3 or 4 carbon atoms;

R(70) and R(71) are independently hydrogen, alkyl having 1, 2, 3, 4, 5, 6, 7 or 8 carbon atoms, -Q(3)-phenyl, which is unsubstituted or substituted by 1, 2 or 3 identical or different substituents selected from alkyl having 1, 2, 3, 4, 5, 6, 7 or 8 carbon atoms, F, Cl, Br, I, CF₃, —SOₚR(72), —OR(73), —NR(74)R(75), —CN, —NO₂, —CO—R(76), and alkenyl having 2, 3, 4, 5, 6, 7 or 8 carbon atoms;

R(63) and R(72) are independently alkyl having 1, 2, 3 or 4 carbon atoms or —NR(77)R(78);

R(64), R(65), R(66), R(68), R(69), R(73), R(74), R(75), R(77) and R(78) are independently hydrogen or alkyl having 1, 2, 3 or 4 carbon atoms;

R(67) and R(76) are independently hydrogen, alkyl having 1, 2, 3, 4, 5, 6, 7 or 8 carbon atoms or —OR(89);

R(79) is alkyl having 1, 2, 3, 4, 5, 6, 7 or 8 carbon atoms, —NR(82)R(83) or phenyl, which is unsubstituted or substituted by 1, 2 or 3 identical or different substituents selected from F, Cl, Br, I, CF₃, methyl, methoxy, hydroxyl and —NR(82)R(83);

R(80) is hydrogen, alkyl having 1, 2, 3, 4, 5, 6, 7 or 8 carbon atoms or —OR(84);

R(81) is hydrogen, alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl and —NR(82)R(83);

R(82) and R(83) are independently hydrogen or alkyl having 1, 2, 3 or 4 carbon atoms;

R(84) is hydrogen or alkyl having 1, 2, 3, 4, 5, 6, 7 or 8 carbon atoms;

R(85) is hydrogen, alkyl having 1, 2, 3, 4, 5, 6, 7 or 8 carbon atoms or —CO—R(86);

R(86) is alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, and hydroxyl;

R(87) and R(88) are independently alkyl having 1, 2, 3, 4, 5, 6, 7 or 8 carbon atoms, cycloalkyl having 3, 4, 5, 6, 7 or 8 carbon atoms or phenyl, which is unsubstituted or substituted by 1, 2 or 3 identical or different substituents selected from F, Cl, Br, I, CF₃, methyl, methoxy or hydroxyl;

R(89) is hydrogen or alkyl having 1, 2, 3, 4, 5, 6, 7 or 8 carbon atoms;

R(92) and R(93) independently of one another -(alkylenyl having 2, 3, or 4 carbon atoms)-O—R(94);

R(94) is hydrogen or alkyl having 1, 2, 3, 4, 5, 6, 7 or 8 carbon atoms;

R(95) is hydrogen or alkyl having 1, 2, 3, 4, 5, 6, 7 or 8 carbon atoms;

Z is carbonyl or sulfonyl;

A and B are independently aryl, which has 6, 7, 8, 9, 10, 11, 12, 13 or 14 carbon atoms, preferably phenyl, 1-naphthyl or 2-naphthyl, and is unsubstituted or substituted by 1, 2 or 3 identical or different substituents selected from alkyl having 1, 2, 3, 4, 5, 6, 7 or 8 carbon atoms, F, Cl, Br, I, CF₃, —SOₙR(63), —OR(64), —NR(65)R(66), —CN, —NO₂ and —CO—R(67), heteroaryl having 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(68)R(69), cycloalkyl having 3, 4, 5, 6 or 7 carbon atoms, —O—R(70), or —O—R(71);

Y is a covalent bond, CR(16)R(17), CO, S, SO₂, O or NR(18);

a is zero, 1 or 2

b is zero, 1 or 2;

c is zero, 1 or 2;

d is zero, 1 or 2;

e is zero, 1 or 2;

f is zero, 1 or 2;

g is zero, 1 or 2;

i is zero, 1 or 2;

j is zero, 1 or 2;

k is zero, 1 or 2;

n is zero, 1 or 2;

p is are independently zero, 1 or 2;

q are independently zero, 1, or 2;

Q(1) is a covalent bond, alkylenyl having 1, 2, 3 or 4 carbon atoms, or alkenylenyl having 2, 3 or 4 carbon atoms;

Q(2) is a covalent bond, alkylenyl having 1, 2, 3 or 4 carbon atoms, or alkenylenyl having 2, 3 or 4 carbon atoms; and

Q(3) is a covalent bond, alkylenyl having 1, 2, 3 or 4 carbon atoms, or alkenylenyl having 2, 3 or 4 carbon atoms.

The invention further relates to the method wherein the cellular sodium-dependent chloride/bicarbonate exchanger inhibitor is a compound of the formula I wherein

X is

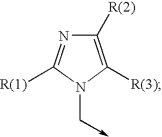

R(1) is alkyl having 1, 2, 3 or 4 carbon atoms or phenyl, which is unsubstituted or substituted by a substituent selected from F, Cl, CF₃, methyl or methoxy;

R(2) and R(3) are independently hydrogen, F, Cl, CF₃, —CN, CO—R(24), —O—R(25), alkyl having 1, 2, 3 or 4 carbon atoms, cycloalkyl having 3, 4, 5, 6 or 7 carbon atoms; or phenyl, which is unsubstituted or substituted by a substituent selected from F, Cl, CF₃, methyl or methoxy; heteroaryl having 1, 2, 3, 4, 5, 6, 7, 8 or 9 carbon atoms and is unsubstituted or substituted by a substituent selected from F, Cl, CF₃, methyl and methoxy, or —SO_{f}—R(37);

R(13), R(14) and R(15) are independently hydrogen, methyl, F, Cl, CF₃, —CN, —SO₂—R(79), —CO—R(80) or —O—R(81); and

R(24) is hydrogen, alkyl having 1, 2, 3 or 4 carbon atoms, OR(26) or phenyl, which is unsubstituted or substituted by a substituent selected from F, Cl, CF₃, methyl and methoxy;

R(25) is hydrogen, alkyl having 1, 2, 3 or 4 carbon atoms, phenyl, which is unsubstituted or substituted by a substituent selected from F, Cl, CF₃, methyl and methoxy, or heteroaryl which is unsubstituted or substituted by a substituent selected from F, Cl, CF₃, methyl and methoxy;

R(26) is hydrogen, methyl or ethyl;

R(37) is alkyl having 1, 2, 3 or 4 carbon atoms or phenyl, which is unsubstituted or substituted by a substituent selected from F, Cl, CF₃, methyl and methoxy;

R(79) and R(81) are independently alkyl having 1, 2, 3 or 4 carbon atoms or phenyl, which is unsubstituted or substituted by a substituent selected from F, Cl, CF₃, methyl and methoxy;

R(80) is hydrogen, methyl or —OR(84);

R(84) is hydrogen or alkyl having 1, 2, 3, or 4 carbon atoms;

Y is a covalent bond or methylene; and

f is zero or 2.

The invention further relates to the method wherein the cellular sodium-dependent chloride/bicarbonate exchanger inhibitor is a compound of the formula II

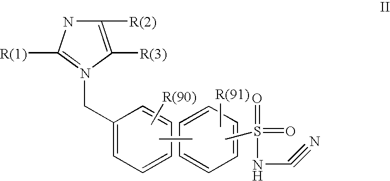

or a stereoisomeric form thereof or a mixture of stereoisomeric forms in any ratio, or a physiologically tolerated salt thereof,

wherein:

R(1) is hydrogen, alkyl having 1, 2, 3, 4, 5, 6, 7 or 8 carbon atoms, —CₐH₂ₐ-Phenyl, which phenyl is unsubstituted or substituted by 1, 2 or 3 identical or different substituents selected from F, Cl, Br, I, CF₃, methyl, methoxy, hydroxyl and —NR(19)R(20), —C_{b}H_{2b}-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(21)R(22), or —C_{c}H_{2c}-cycloalkyl, which cycloalkyl has 3, 4, 5, 6 or 7 carbon atoms;

R(2) and R(3) are independently hydrogen, F, Cl, Br, I, CF₃, —CN, —NO₂, —CH₂OR(23), —CO—R(24), O—R(25), —O-(alkylenyl having 2, 3, or 4 carbon atoms)-O—R(23), or —NR(92)R(93), alkyl having 1, 2, 3, 4, 5, 6, 7 or 8 carbon atoms, cycloalkyl having 3, 4, 5, 6 or 7 carbon atoms or —C_{d}H_{2d}-phenyl, which phenyl is unsubstituted or substituted by 1, 2 or 3 identical or different substituents selected from F, Cl, Br, I, CF₃, methyl, methoxy, hydroxyl and —NR(33)R(34), —CₑH₂ₑ-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(35)R(36), or —SO_{f}—R(37);

R(19) and R(20) are independently hydrogen or alkyl having 1, 2, 3 or 4 carbon atoms;

R(21) and R(22) are independently hydrogen or alkyl having 1, 2, 3 or 4 carbon atoms;

R(23) is hydrogen or alkyl having 1, 2, 3, 4, 5, 6, 7 or 8 carbon atoms

R(24) is hydrogen, alkyl having 1, 2, 3, 4, 5, 6, 7 or 8 carbon atoms, —OR(26) or phenyl, which is unsubstituted or substituted by 1, 2 or 3 identical or different substituents selected from F, Cl, Br, I, CF₃, methyl, methoxy, hydroxyl and —NR(31)R(32);

R(25) is hydrogen, alkyl having 1, 2, 3, 4, 5, 6, 7 or 8 carbon atoms, phenyl, which is unsubstituted or substituted by 1, 2 or 3 identical or different substituents selected from F, Cl, Br, I, CF₃, methyl, methoxy, hydroxyl and —NR(29)R(30), or heteroaryl which has 1, 2, 3, 4, 5, 6, 7, 8 or 9 carbon atoms and is unsubstituted or substituted by 1, 2 or 3 identical or different substituents selected from F, Cl, Br, I, CF₃, CH₃, methoxy, hydroxyl and —NR(31)R(32);

R(26) is hydrogen or alkyl having 1, 2, 3, 4, 5, 6, 7 or 8 carbon atoms;

R(27) and R(28) are independently hydrogen or alkyl having 1, 2, 3 or 4 carbon atoms;

R(29) and R(30) are independently hydrogen or alkyl having 1, 2, 3 or 4 carbon atoms;

R(31) and R(32) are independently hydrogen or alkyl having 1, 2, 3 or 4 carbon atoms;

R(33) and R(34) are independently hydrogen or alkyl having 1, 2, 3 or 4 carbon atoms;

R(35) and R(36) are independently hydrogen or alkyl having 1, 2, 3 or 4 carbon atoms;

R(37) is alkyl having 1, 2, 3, 4, 5, 6, 7 or 8 carbon atoms, cycloalkyl having 3, 4, 5, 6 or 7 carbon atoms or —C_{g}H_{2g}-phenyl, which phenyl is unsubstituted or substituted by 1, 2 or 3 identical or different substituents selected from the group consisting of F, Cl, Br, I, CF₃, methyl, methoxy, hydroxyl and —NR(38)R(39);

R(38) and R(39) are independently hydrogen or alkyl having 1, 2, 3 or 4 carbon atoms;

R(79) is alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl and —NR(82)R(83);

R(80) is hydrogen, alkyl having 1, 2, 3, 4, 5, 6, 7 or 8 carbon atoms or —OR(84);

R(81) is hydrogen, alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl and —NR(82)R(83);

R(82) and R(83) are independently hydrogen or alkyl having 1, 2, 3 or 4 carbon atoms;

R(84) is hydrogen or alkyl having 1, 2, 3, 4, 5, 6, 7 or 8 carbon atoms;

R(90) and R(91) are independently hydrogen, alkyl having 1, 2, 3, 4, 5, 6, 7 or 8 carbon atoms, F, Cl, Br, I, CF₃, —CN, —NO₂, —SO_{q}—R(79), —CO—R(80), —O—R(81) or —O-(alkylenyl having 2, 3 or 4 carbon atoms)-O—R(95);

R(92) and R(93) are independently -(alkylenyl having 2, 3, or 4 carbon atoms)-O—R(94);

R(94) is hydrogen or alkyl having 1, 2, 3, 4, 5, 6, 7 or 8 carbon atoms;

R(95) is hydrogen or alkyl having 1, 2, 3, 4, 5, 6, 7 or 8 carbon atoms;

a is zero, 1 or 2;

b is zero, 1 or 2;

c is zero, 1 or 2;

d is zero, 1 or 2;

e is zero, 1 or 2;

f is zero, 1 or 2;

g is zero, 1 or 2; and

q is zero, 1 or 2.

The invention further relates to the method wherein the cellular sodium-dependent chloride/bicarbonate exchanger inhibitor is a compound of the formula II wherein

R(1) is hydrogen, alkyl having 1, 2, 3, 4, 5, 6, 7 or 8 carbon atoms, —CₐH₂ₐ-phenyl, which phenyl is unsubstituted or substituted by 1, 2 or 3 identical or different substituents selected from F, Cl, Br, I, CF₃, methyl, methoxy, hydroxyl and —NR(19)R(20), —C_{b}H_{2b}-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(21)R(22) or —C_{c}H_{2c}-cycloalkyl, which cycloalkyl has 3, 4, 5, 6 or 7 carbon atoms;

R(2) and R(3) are independently hydrogen, F, Cl, Br, I, CF₃, —CN, —NO₂, CH₂OR(23), CO—R(24), —O—R(25), alkyl having 1, 2, 3, 4, 5, 6, 7 or 8 carbon atoms, cycloalkyl having 3, 4, 5, 6 or 7 carbon atoms or —C_{d}H_{2d}-phenyl, which phenyl is unsubstituted or substituted by 1, 2 or 3 identical or different substituents selected from F, Cl, Br, I, CF₃, methyl, methoxy, hydroxyl and —NR(33)R(34), —CₑH₂ₑ-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(35)R(36), or —SO_{f}—R(37);

R(19) and R(20) are independently hydrogen or alkyl having 1, 2, 3 or 4 carbon atoms;

R(21) and R(22) are independently hydrogen or alkyl having 1, 2, 3 or 4 carbon atoms;

R(23) is hydrogen or alkyl having 1, 2, 3, 4, 5, 6, 7 or 8 carbon atoms;

R(24) is hydrogen, alkyl having 1, 2, 3, 4, 5, 6, 7 or 8 carbon atoms, —OR(26) or phenyl, which is unsubstituted or substituted by 1, 2 or 3 identical or different substituents selected from F, Cl, Br, I, CF₃, methyl, methoxy, hydroxyl and —NR(31)R(32);

R(25) is hydrogen, alkyl having 1, 2, 3, 4, 5, 6, 7 or 8 carbon atoms, phenyl, which is unsubstituted or substituted by 1, 2 or 3 identical or different substituents selected from F, Cl, Br, I, CF₃, methyl, methoxy, hydroxyl and —NR(29)R(30), or heteroaryl, which has 1, 2, 3, 4, 5, 6, 7, 8 or 9 carbon atoms and is unsubstituted or substituted by 1, 2 or 3 identical or different substituents selected from F, Cl, Br, I, CF₃, CH₃, methoxy, hydroxyl and —NR(31)R(32);

R(26) is hydrogen or alkyl having 1, 2, 3, 4, 5, 6, 7 or 8 carbon atoms;

R(29) and R(30) are independently hydrogen or alkyl having 1, 2, 3 or 4 carbon atoms;

R(31) and R(32) are independently hydrogen or alkyl having 1, 2, 3 or 4 carbon atoms;

R(33) and R(34) are independently hydrogen or alkyl having 1, 2, 3 or 4 carbon atoms;

R(35) and R(36) are independently hydrogen or alkyl having 1, 2, 3 or 4 carbon atoms;

R(37) is alkyl having 1, 2, 3, 4, 5, 6, 7 or 8 carbon atoms, cycloalkyl having 3, 4, 5, 6 or 7 carbon atoms or —C_{g}H_{2g}-phenyl, which phenyl is unsubstituted or substituted by 1, 2 or 3 identical or different substituents selected from the group consisting of F, Cl, Br, I, CF₃, methyl, methoxy, hydroxyl and —NR(38)R(39);

R(38) and R(39) are independently hydrogen or alkyl having 1, 2, 3 or 4 carbon atoms;

R(79) is alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl and NR(82)R(83);

R(82) and R(83) are independently hydrogen or alkyl having 1, 2, 3 or 4 carbon atoms;

R(91) is hydrogen,

R(90) is —SO_{q}—R(79),

a is zero, 1 or 2;

b is zero, 1 or 2;

c is zero, 1 or 2;

d is zero, 1 or 2;

e is zero, 1 or 2;

f is zero, 1 or 2;

g is zero, 1 or 2; and

q is zero, 1 or 2.

The invention further relates to the method wherein the cellular sodium-dependent chloride/bicarbonate exchanger inhibitor is a compound of the formula II wherein

R(1) is —CₐH₂ₐ-phenyl, which phenyl is unsubstituted or substituted by 1 or 2 identical or different substituents selected from F, Cl, Br, CF₃, methyl, methoxy, hydroxyl or —NR(19)R(20);

R(2) is F, Cl, Br, I or OR(25), in particular Cl;

R(3) is CO—R(24);

R(19) and R(20) are independently hydrogen or methyl;

R(24) and R(91) are hydrogen;

R(25) is alkyl having 1, 2, 3 or 4 carbon atoms;

R(79) equal to alkyl having 1, 2, 3 or 4 carbon atoms;

R(90) is SO₂R(79); and

a is zero, 1 or 2.

The invention further relates to the method wherein the cellular sodium-dependent

chloride/bicarbonate exchanger inhibitor is a compound of the formula II wherein

R(1) is —CₐH₂ₐ-phenyl, which phenyl is unsubstituted or substituted by 1 or 2 identical or different substituents selected from F, Cl, Br, CF₃, methyl, methoxy, hydroxyl or —NR(19)R(20);

R(2) is F, Cl, Br, I or OR(25), in particular Cl;

R(3) is CO—R(24);

R(19) and R(20) are independently hydrogen or methyl;

R(24) and R(91) are hydrogen;

R(25) is alkyl having 1, 2, 3 or 4 carbon atoms;

R(79) equal to alkyl having 1, 2, 3 or 4 carbon atoms;

R(90) is SO₂R(79); and

a is zero, 1 or 2.

The invention further relates to the method wherein the compound of the formula II is a compound of the formula IIa or IIb

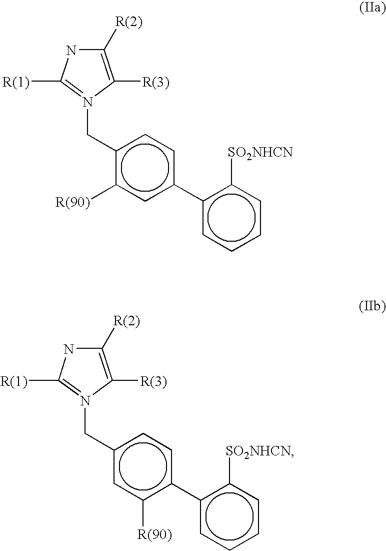

where the radicals R(1), R(2), R(3) and R(90) are as defined for formula II.

The invention further relates to the method wherein the cellular sodium-dependent chloride/bicarbonate exchanger inhibitor is a compound of the formula II wherein

R(1) is alkyl having 1, 2, 3, 4, 5, 6, 7 or 8 carbon atoms, —CₐH₂ₐ-phenyl, which phenyl is unsubstituted or substituted by 1, 2 or 3 identical or different substituents selected from F, Cl, Br, I, CF₃, methyl, methoxy, hydroxyl and —NR(19)R(20), —C_{b}H_{2b}-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(21)R(22), or; or —C_{c}H_{2c}-cycloalkyl, which cycloalkyl has 3, 4, 5, 6 or 7 carbon atoms;

R(2) and R(3) are independently hydrogen, F, Cl, Br, I, CF₃, —CN, —NO₂, —CH₂OR(23), —CO—R(24), —O—R(25), —O-(alkylenyl having 2, 3, or 4 carbon atoms)-O—R(23), —NR(92)R(93), alkyl having 1, 2, 3, 4, 5, 6, 7 or 8 carbon atoms, cycloalkyl having 3, 4, 5, 6 or 7 carbon atoms, —C_{d}H_{2d}-phenyl, which phenyl is unsubstituted or substituted by 1, 2 or 3 identical or different substituents selected from F, Cl, Br, I, CF₃, methyl, methoxy, hydroxyl or —NR(33)R(34), —C_{c}H_{2c}-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl or —NR(35)R(36), or —SO_{f}—R(37), and at least one of R(2) or R(3) is —O-(alkylenyl having 2, 3, or 4 carbon atoms)-O—R(23) or —NR(92)R(93);

R(19) and R(20) are independently hydrogen or alkyl having 1, 2, 3 or 4 carbon atoms;

R(21) and R(22) are independently hydrogen or alkyl having 1, 2, 3 or 4 carbon atoms;

R(23) is hydrogen or alkyl having 1, 2, 3, 4, 5, 6, 7 or 8 carbon atoms

R(24) is hydrogen, alkyl having 1, 2, 3, 4, 5, 6, 7 or 8 carbon atoms, —OR(26) or phenyl, which is unsubstituted or substituted by 1, 2 or 3 identical or different substituents selected from F, Cl, Br, I, CF₃, methyl, methoxy, hydroxyl and —NR(27)R(28);

R(25) hydrogen, alkyl having 1, 2, 3, 4, 5, 6, 7 or 8 carbon atoms, or phenyl, which is unsubstituted or substituted by 1, 2 or 3 identical or different substituents selected from F, Cl, Br, I, CF₃, methyl, methoxy, hydroxyl or —NR(29)R(30), or heteroaryl, which has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl or —NR(31)R(32);

R(26) is hydrogen or alkyl having 1, 2, 3, 4, 5, 6, 7 or 8 carbon atoms;

R(27) and R(28) are independently hydrogen or alkyl having 1, 2, 3 or 4 carbon atoms;

R(29) and R(30) are independently hydrogen or alkyl having 1, 2, 3 or 4 carbon atoms;

R(31) and R(32) are independently hydrogen or alkyl having 1, 2, 3 or 4 carbon atoms; or

R(33) and R(34) are independently hydrogen or alkyl having 1, 2, 3 or 4 carbon atoms;

R(35) and R(36) are independently hydrogen or alkyl having 1, 2, 3 or 4 carbon atoms;

R(37) is alkyl having 1, 2, 3, 4, 5, 6, 7 or 8 carbon atoms, cycloalkyl having 3, 4, 5, 6 or 7 carbon atoms or —C_{g}H_{2g}-phenyl which is unsubstituted or substituted by 1, 2 or 3 identical or different substituents selected from F, Cl, Br, I, CF₃, methyl, methoxy, hydroxyl or —NR(38)R(39);

R(38) and R(39) are independently hydrogen or alkyl having 1, 2, 3 or 4 carbon atoms;

R(79) alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl or —NR(82)R(83);

R(82) and R(83) are independently hydrogen or alkyl having 1, 2, 3, or 4 carbon atoms;

R(80) is hydrogen, alkyl having 1, 2, 3, 4, 5, 6, 7 or 8 carbon atoms or OR(84);

R(84) is hydrogen, alkyl having 1, 2, 3, 4, 5, 6, 7 or 8 carbon atoms;

R(81) is hydrogen, alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl or —NR(82)R(83);

R(82) and R(83) are independently hydrogen or alkyl having 1, 2, 3 or 4 carbon atoms;

R(90) and R(91) are independently hydrogen, alkyl having 1, 2, 3, 4, 5, 6, 7 or 8 carbon atoms, F, Cl, Br, I, CF₃, —CN, —NO₂, —SO_{q}—R(79), —CO—R(80), —O—R(81) or —O-(alkylenyl having 2, 3 or 4 carbon atoms)-O—R(95);

R(92) and R(93) are independently -(alkylenyl having 2, 3, or 4 carbon atoms)-O—R(94);

R(94) is hydrogen or alkyl having 1, 2, 3, 4, 5, 6, 7 or 8 carbon atoms;

R(95) is hydrogen or alkyl having 1, 2, 3, 4, 5, 6, 7 or 8 carbon atoms;

a is zero, 1 or 2;

b is zero, 1 or 2;

c is zero, 1 or 2;

d is zero, 1 or 2;

f is zero, 1 or 2;

g is zero, 1 or 2; and

q is zero, 1 or 2.

The invention further relates to the method wherein the cellular sodium-dependent chloride/bicarbonate exchanger inhibitor is 4′-[5-formyl-4-(2-methoxyethoxy)-2-phenyl-1-imidazolylmethyl]-3′-methylsulfonylbiphenyl-2-sulfonylcyanamide of the following formula

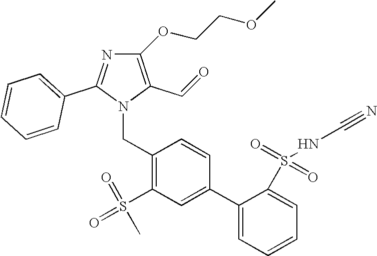

or 4′-{[benzyl(thiophene-2-sulfonyl)amino]methyl}-3′-methanesulfonylbiphenyl-2-sulfonylcyanamide of the following formula

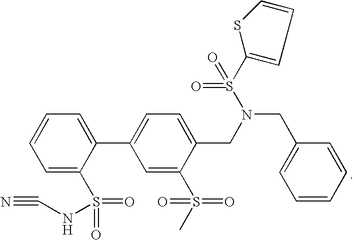

The invention further relates to the method wherein the cellular sodium-dependent chloride/bicarbonate exchanger inhibitor is a compound of the formula III

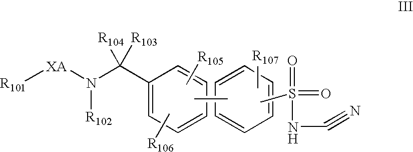

or a stereoisomeric form thereof or a mixture of stereoisomeric forms in any ratio, or a physiologically tolerated salt thereof,

wherein:

R(101) is alkyl, which has 1, 2, 3, 4, 5, 6, 7 or 8 carbon atoms and is optionally substituted by fluorine at one to all of the hydrogen atoms thereof, alkenyl having 2, 3, 4, 5, 6, 7, 8, 9, 10, 11 or 12 carbon atoms; -Q(4)—Y,

R(102) is hydrogen; alkyl, which has 1, 2, 3, 4, 5, 6, 7 or 8 carbon atoms and is optionally substituted by fluorine at one to all of the hydrogen atoms thereof, alkenyl having 2, 3, 4, 5, 6, 7, 8, 9, 10, 11 or 12 carbon atoms, alkynyl having 2, 3, 4, 5, 6, 7 or 8 carbon atoms, -Q(5)—Y,

R(103) and R(104) are independently hydrogen or alkyl having 1, 2, 3, 4, 5, 6, 7 or 8 carbon atoms;

R(105), R(106) and R(107) are independently hydrogen, alkyl having 1, 2, 3, 4, 5, 6, 7 or 8 carbon atoms, F, Cl, Br, I, CF₃, —CN, —NO₂, —SO_{q}—R(79), —CO—R(80), —O—R(81), or —O-(alkylenyl having 2, 3, or 4 carbon atoms)-O—R(95);

R(70) and R(71) are independently hydrogen, alkyl having 1, 2, 3, 4, 5, 6, 7 or 8 carbon atoms, -Q(3)-phenyl, which is unsubstituted or substituted by 1, 2 or 3 identical or different substituents selected from alkyl having 1, 2, 3, 4, 5, 6, 7 or 8 carbon atoms, F, Cl, Br, I, CF₃, —SOₚR(72), —OR(73), —NR(74)R(75), —CN, —NO₂, —CO—R(76), and alkenyl having 2, 3, 4, 5, 6, 7 8, 9, 10, 11 or 12 carbon atoms;

R(72) is alkyl, which has 1, 2, 3 or 4 carbon atoms and is optionally substituted by fluorine at one to all of the hydrogen atoms thereof, preferably CF₃, or —NR(77)R(78);

R(68), R(69), R(73), R(74), R(75) R(77) and R(78) are independently hydrogen or alkyl having 1, 2, 3 or 4 carbon atoms;

(76) is hydrogen, alkyl having 1, 2, 3, 4, 5, 6, 7 or 8 carbon atoms or —OR(89);

R(79) is alkyl having 1, 2, 3, 4, 5, 6, 7 or 8 carbon atoms, —NR(82)R(83) or phenyl which is unsubstituted or substituted by 1, 2 or 3 identical or different substituents selected from F, Cl, Br, I, CF₃, methyl, methoxy, hydroxyl and —NR(82)R(83);

R(80) is hydrogen, alkyl having 1, 2, 3, 4, 5, 6, 7 or 8 carbon atoms or OR(84);

R(81) hydrogen, alkyl having 1, 2, 3, 4, 5, 6, 7 or 8 carbon atoms, optionally substituted by (C₁-C₄)-alkoxy, or phenyl which is unsubstituted or substituted by 1, 2 or 3 identical or different substituents selected from F, Cl, Br, I, CF₃, methyl, methoxy, hydroxyl or NR(82)R(83);

R(82), R(83), R(108) and R(109) are independently hydrogen, alkyl having 1, 2, 3 or 4 carbon atoms or (C₁-C₄)-alkanoyl, preferably acetyl;

R(84) is hydrogen or alkyl having 1, 2, 3, 4, 5, 6, 7 or 8 carbon atoms;

R(89) is hydrogen or alkyl having 1, 2, 3, 4, 5, 6, 7 or 8 carbon atoms;

R(95) is hydrogen or alkyl having 1, 2, 3, 4, 5, 6, 7 or 8 carbon atoms;

R(96) and R(97) are independently hydrogen or CO—OR(99);

R(98) is hydrogen, alkyl, which has 1, 2, 3 or 4 carbon atoms, and is optionally substituted by fluorine at one to all of the hydrogen atoms thereof, preferably CF₃, or optionally substituted by alkoxy, aryl having 6, 7, 8, 9, 10, 11, 12, 13 or 14 carbon atoms, preferably phenyl, 1-naphthyl or 2-naphthyl, and is optionally substituted by 1, 2 or 3 identical or different substituents selected from F, Cl, Br, I, CF₃,

NR(108)R(109), —CN, and —NO2;

R(99) hydrogen, alkyl having 1, 2, 3, 4, 5, 6, 7 or 8 carbon atoms or —CᵣH₂ᵣ-phenyl;

n is zero, 1 or 2;

p is zero, 1 or 2;

q is zero, 1 or 2;

r is equal to 1, 2, 3 or 4;

XA is carbonyl, —CO—NH—, —CO—CO— or sulfonyl;

Y and Z are independently aryl, which has 6, 7, 8, 9, 10, 11, 12, 13 or 14 carbon atoms, preferably phenyl, 1-naphthyl or 2-naphthyl, which is unsubstituted or substituted by 1, 2, 3, 4 or 5 identical or different substituents selected from alkyl having 1, 2, 3, 4, 5, 6, 7 or 8 carbon atoms, aryl having 6, 7, 8, 9, 10, 11, 12, 13 or 14 carbon atoms, preferably phenyl, 1-naphthyl or 2-naphthyl, F, Cl, Br, I, CF₃, SOₙR(72), —OR(73), —NR(74)R(75), —CN, —NO₂ and —CO—R(76), or where two substituents together form a fused heterocyclyl substitute, preferably methylenedioxy, heteroaryl, which has 1, 2, 3, 4, 5, 6, 7, 8 or 9 carbon atoms and is substituted by 1, 2 or 3 identical or different substituents selected from F, Cl, Br, I, CF₃, methyl, methoxy, hydroxyl and —NR(68)R(69), cycloalkyl, which has 3, 4, 5, 6, 7, 8, 9 or 10 carbon atoms, preferably cyclopropyl, cyclopentyl, cyclohexyl, 1, 2, 3,4-tetrahydronaphthyl or indanyl, and is optionally substituted by aryl having 6, 7, 8, 9, 10, 11, 12, 13 or 14 carbon atoms, preferably phenyl, 1-naphthyl or 2-naphthyl, —O—R(70), —O—R(71), —SO₂—R(70), arylalkylcarbonyl, preferably phenyl-CH₂—CO—, or heterocyclyl;

Q(3) is a covalent bond, alkylenyl having 1, 2, 3 or 4 carbon atoms, or alkenylenyl having 2, 3 or 4 carbon atoms;

Q(4) is a covalent bond, alkylenyl having 1, 2, 3 or 4 carbon atoms, or alkenylenyl having 2, 3 or 4 carbon atoms, and 1, 2 or 3 hydrogen atoms in Q(4) are optionally substituted independently by a substituent selected from aryl having 6, 7, 8, 9, 10, 11, 12, 13 or 14 carbon atoms, preferably phenyl, 1-naphthyl or 2-naphthyl, amino, —NR(96)R(97), alkoxycarbonyl, COOR(98), alkyl having 1, 2, 3 or 4 carbon atoms or (C₆-C₁₄)-aryl-(C₁-C₄)-alkylcarbonyl, preferably phenylacetyl; and

Q(5) is a covalent bond, alkylenyl having 1, 2, 3 or 4 carbon atoms, or alkenylenyl having 2, 3 or 4 carbon atoms, and 1, 2 or 3 hydrogen atoms in Q(5) are optionally substituted independently by a substituent selected from aryl having 6, 7, 8, 9, 10, 11, 12, 13 or 14 carbon atoms, preferably phenyl, 1-naphthyl or 2-naphthyl, amino, —NR(96)R(97), —(C₁-C₄)-alkoxycarbonyl, —COOR(98), or alkyl having 1, 2, 3 or 4 carbon atoms.

The invention further relates to the method the cellular sodium-dependent chloride/bicarbonate exchanger inhibitor is a compound of the formula IV

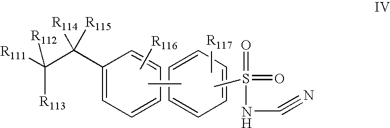

or a stereoisomeric form thereof or a mixture of stereoisomeric forms in any ratio, or a physiologically tolerated salt thereof, wherein:

R(111) is hydrogen, alkyl having 1, 2, 3, 4, 5, 6, 7 or 8 carbon atoms, 1-naphthyl, 2-naphthyl, —CᵢH₂ᵢ-cycloalkyl having 3, 4, 5, 6 or 7 carbon atoms or —CᵢH₂ᵢ-phenyl, where the phenyl moiety is unsubstituted or substituted by 1, 2 or 3 identical or different substituents selected from alkyl having 1, 2, 3, 4, 5, 6, 7 or 8 carbon atoms, F, Cl, Br, I, CF₃, SOⱼR(48), OR(49), NR(50)R(51), —CN, —NO₂ or CO—R(52), or

R(111) and R(113) taken together with the carbon atom bearing them form cycloalkyl having 3, 4, 5, 6 or 7 carbon atoms or fluorenyl;

R(112), R(113), R(114) and R(115) are independently hydrogen, F, CF₃, O—R(56), alkyl having 1, 2, 3, 4, 5, 6, 7 or 8 carbon atoms, cycloalkyl having 3, 4, 5, 6 or 7 carbon atoms, —CₖH₂ₖ-phenyl, which phenyl is unsubstituted or substituted by 1, 2 or 3 identical or different substituents selected from the group F, Cl, Br, I, CF₃, methyl, methoxy, hydroxyl or NR(57)R(58);

R(112) and R(114) together a second bond between the carbon atoms bearing them;

R(116) and R(117) are independently hydrogen, alkyl having 1, 2, 3, 4, 5, 6, 7 or 8 carbon atoms, F, Cl, Br, I, CF₃, —CN, —NO₂, —SO_{q}—R(79), —CO—R(80), —O—R(81), or —O-(alkylenyl having 2, 3 or 4 carbon atoms)-O—R(95);

R(48) is alkyl having 1, 2, 3 or 4 carbon atoms or —NR(53)R(54);

R(49) hydrogen or alkyl having 1, 2, 3 or 4 carbon atoms;

R(50) and R(51) are independently hydrogen or alkyl having 1, 2, 3 or 4 carbon atoms;

R(52) is hydrogen, alkyl having 1, 2, 3, 4, 5, 6, 7 or 8 carbon atoms or OR(55);

R(53) and R(54) are independently hydrogen or alkyl having 1, 2, 3 or 4 carbon atoms;

R(55) is hydrogen, alkyl having 1, 2, 3, 4, 5, 6, 7 or 8 carbon atoms;

R(56) is hydrogen, alkyl having 1, 2, 3, 4, 5, 6, 7 or 8 carbon atoms, phenyl, which is unsubstituted or substituted by 1, 2 or 3 identical or different substituents selected from the group of F, Cl, Br, I, CF₃, methyl, methoxy, hydroxyl or NR(59)R(60), or heteroaryl, which has 1, 2, 3, 4, 5, 6, 7, 8 or 9-carbon atoms, and is unsubstituted or substituted by 1, 2 or 3 identical or different substituents selected from the group of F, Cl, Br, I, CF₃, methyl, methoxy, hydroxyl or NR(61)R(62);

R(57) and R(58) are independently hydrogen or alkyl having 1, 2, 3 or 4 carbon atoms;

R(59) and R(60) are independently hydrogen or alkyl having 1, 2, 3 or 4 carbon atoms;

R(61) and R(62) are independently hydrogen or alkyl having 1, 2, 3 or 4 carbon atoms;

R(79) is alkyl having 1, 2, 3, 4, 5, 6, 7 or 8 carbon atoms, —NR(82)R(83), phenyl, which is unsubstituted or substituted by 1, 2 or 3 identical or different substituents selected from the group of F, Cl, Br, I, CF₃, methyl, methoxy, hydroxyl or —NR(82)R(83);

R(80) is hydrogen, alkyl having 1, 2, 3, 4, 5, 6, 7 or 8 carbon atoms or OR(84);

R(84) and R(95) are indendently hydrogen, alkyl having 1, 2, 3, 4, 5, 6, 7 or 8 carbon atoms;

R(81) is hydrogen, alkyl having 1, 2, 3, 4, 5, 6, 7 or 8 carbon atoms or phenyl which is unsubstituted or substituted by 1, 2 or 3 identical or different substituents selected from the group of F, Cl, Br, I, CF₃, methyl, methoxy, hydroxyl or NR(82)R(83);

R(82) and R(83) are independently hydrogen or alkyl having 1, 2, 3 or 4 carbon atoms;

i is zero, 1 or 2;

j is zero, 1 or 2;

k is zero, 1 or 2; and

q is zero, 1 or 2.

The invention further relates to the method wherein the cellular sodium-dependent chloride/bicarbonate exchanger inhibitor is a compound of the formula IV wherein

R(111) is methyl, ethyl, 1-naphthyl, 2-naphthyl, cycloalkyl having 3, 4, 5, 6 or 7 carbon atoms or phenyl, which is unsubstituted or substituted by one substituent from the group of alkyl having 1, 2, 3 or 4 carbon atoms, F, Cl, CF₃, —SO₂R(48), —OR(49), —NR(50)R(51), or —CO—R(52), or R(111) and R(113) taken together with the carbon atom bearing them form cycloalkyl having 3, 4, 5, 6 or 7 carbon atoms or fluorenyl;

R(112) and R(114) are hydrogen, or taken together form a second bond between the carbon atoms bearing them;

R(113) and R(115) are independently hydrogen, CF₃, O—R(56), alkyl having 1, 2, 3 or 4 carbon atoms or phenyl which is unsubstituted or substituted by one substituent selected from F, Cl, CF₃, methyl, methoxy, hydroxyl or —NR(57)R(58);

R(116) and R(117) are independently hydrogen, F, Cl, CF₃, —SO₂-methyl, —CO—R(80) or —O—R(81);

R(48) methyl or dimethylamino;

R(49) hydrogen, methyl or ethyl;

R(50) and R(51) are independently hydrogen, methyl or ethyl;

R(52) hydrogen or alkyl having 1, 2, 3 or 4 carbon atoms;

R(57) and R(58) are independently hydrogen, methyl or ethyl;

R(56) is hydrogen, alkyl having 1, 2, 3 or 4 carbon atoms or phenyl which is unsubstituted or substituted by 1 substituent from the group of F, Cl, CF₃, methyl, methoxy, hydroxyl, NR(59)R(60) or heteroaryl having 1, 2, 3, 4, 5, 6, 7, 8 or 9 carbon atoms which is unsubstituted or substituted by one substituent selected from F, Cl, CF₃, methyl, methoxy, hydroxyl or dimethylamino;

R(59) and R(60) are independently hydrogen, methyl or ethyl;

R(80) hydrogen or alkyl having 1, 2, 3 or 4 carbon atoms;

R(81) hydrogen, alkyl having 1, 2, 3 or 4 carbon atoms or pheny, l which is unsubstituted or substituted by 1 substituent from the group of F, Cl, CF₃, methyl, methoxy, hydroxyl or —NR(82)R(83); and

R(82) and R(83) are independently hydrogen, methyl or ethyl.

In particular, the invention is directed to the method wherein the compound of the formula IV in which the biphenyl ring moiety thereof is linked as in the following formula

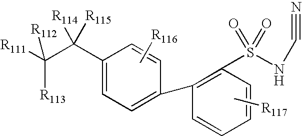

with the sulfonylcyanamide moiety in position 2.

The abovementioned compounds are known and can be prepared as described, for example, in EP 0 855 392, EP 1 097 140, EP 1 097 141, EP 1 076 651, EP 1 053 224 or EP 0 903 339.

The invention further relates to the method further comprising a sodium/hydrogen exchanger inhibitor.

The invention further relates to the method wherein the sodium/hydrogen exchanger inhibitor is a compound of the formula selected from

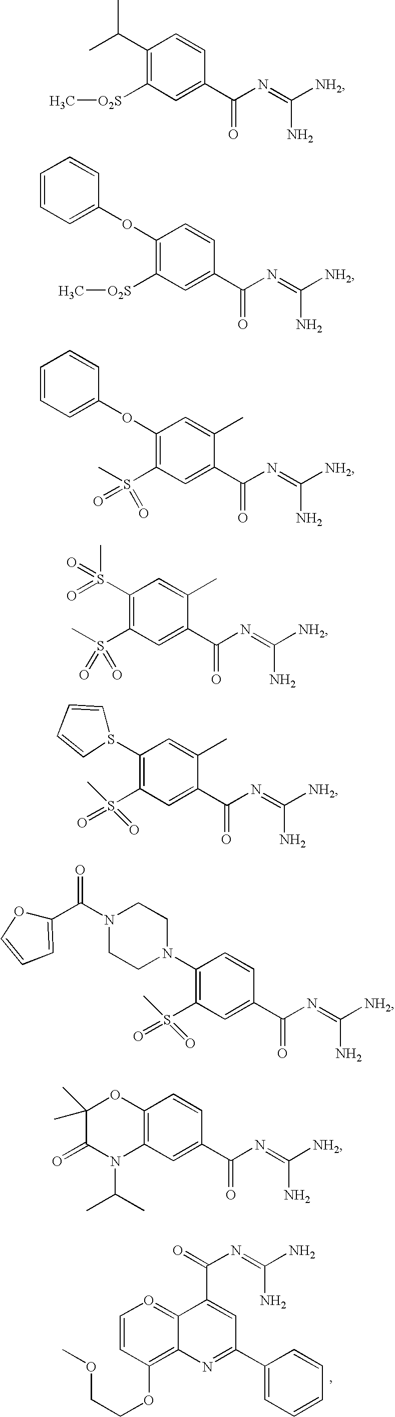

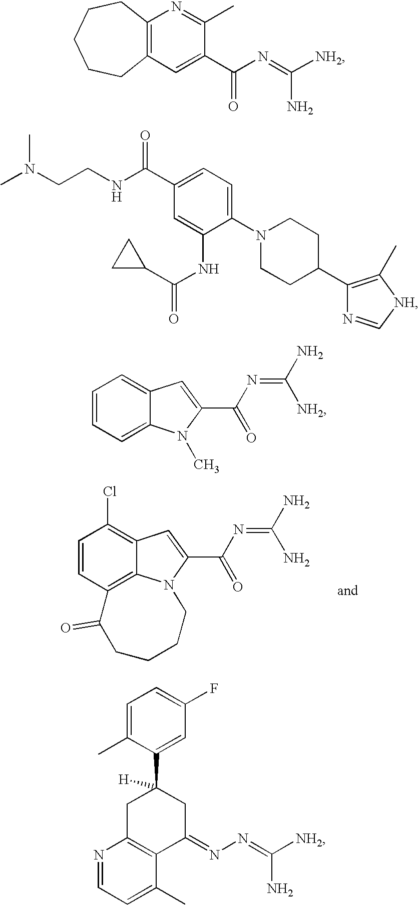

or a physiologically tolerated salt of the sodium/hydrogen exchanger inhibitor.

The invention further relates to the method wherein the sodium/hydrogen exchanger inhibitor is a compound of the formula

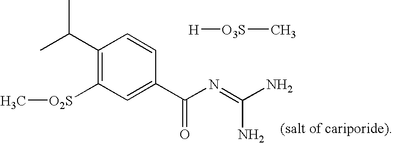

The invention further relates to the method wherein the cellular sodium-dependent chloride/bicarbonate exchanger inhibitor is 4′-[5-formyl-4-(2-methoxyethoxy)-2-phenyl-1-imidazolylmethyl]-3′-methylsulfonylbiphenyl-2-sulfonylcyanamide.

The abovementioned compounds are known and can be prepared as described, for example, in EP 0 416 499, EP 0 556 673, EP 0 589 336, EP 0 622 356, EP 0 699 666, EP 0 708 088, EP 0 719 766, EP 0 726 254, EP 0 787 728, EP 0 972 767, DE 19529612, DE 19601303, WO 99 00379 or T. Kawamoto et al., Eur.J. Pharmacol. 420 (2001), 1-8.

Where the abovementioned compounds allow for diastereoisomeric or enantiomeric forms and result as mixtures thereof in the chosen synthesis, separation into the pure stereoisomers takes place either by chromatography on an optionally chiral support material or, if the racemic abovementioned compounds are able to form salts, by fractional crystallization of the diastereomeric salts formed with an optically active base or acid as aid. Examples of suitable chiral stationary phases for separation of enantiomers by thin-layer or column chromatography are modified silica gel supports (so-called Pirkle phases) and high molecular weight carbohydrates such as triacetylcellulose. Gas chromatographic methods on chiral stationary phases can also be used for analytical purposes after appropriate derivatization known to the skilled worker. To separate enantiomers of the racemic carboxylic acids, diastereomeric salts differing in solubility are formed using an optically active, usually commercially available, base such as (−)-nicotine, (+)- and (−)-phenylethylamine, quinine bases, L-lysine or L- and D-arginine, the less soluble component is isolated as solid, the more soluble diastereomer is deposited from the mother liquor, and the pure enantiomers are obtained from the diastereomeric salts obtained in this way. It is possible in the same way in principle to convert the racemic compounds of the formula I containing a basic group such as an amino group with optically active acids such as (+)-camphor-10-sulfonic acid, D- and L-tartaric acid, D- and L-lactic acid and (+) and (−)-mandelic acid into the pure enantiomers. Chiral compounds containing alcohol or amine functions can also be converted with appropriately activated or, where appropriate, N-protected enantiopure amino acids into the corresponding esters or amides, or conversely convert chiral carboxylic acids with carboxyl-protected enantiopure amino acids into the amides or with enantiopure hydroxy carboxylic acids such as lactic acid into the corresponding chiral esters. The chirality of the amino acid or alcohol residue produced in enantiopure form can then be utilized for separating the isomers by carrying out a separation of the diastereomers which are now present by crystallization or chromatography on suitable stationary phases and then eliminating the included chiral moiety by suitable methods.

Acidic or basic products of the abovementioned compounds can exist in the form of their salts or in free form. Preference is given to pharmacologically suitable salts, e.g., alkali metal or alkaline earth metal salts, or hydrochlorides, hydrobromides, sulfates, hemisulfates, all possible phosphates, and salts of amino acids, natural bases or carboxylic acids.

Physiologically tolerated salts are prepared from the abovementioned compounds able to form salts, including the stereoisomeric forms thereof, in a manner known per se. The carboxylic acids and hydroxamic acids form with basic reagents such as hydroxides, carbonates, bicarbonates, alcoholates and ammonia or organic bases, for example trimethyl- or triethylamine, ethanolamine or triethanolamine or else basic amino acids, for example lysine, ornithine or arginine, stable alkali metal, alkaline earth metal or optionally substituted ammonium salts. Where the abovementioned compounds have basic groups, stable acid addition salts can also be prepared with strong acids. Suitable for this purpose are both inorganic and organic acids, such as hydrochloric, hydrobromic, sulfuric, phosphoric, methanesulfonic, benzenesulfonic, p-toluenesulfonic, 4-bromobenzenesulfonic, cyclohexylsulfamic, trifluoromethylsulfonic, acetic, oxalic, tartaric, succinic or trifluoroacetic acid. Methanesulfonic acid salts of the abovementioned compounds are particularly preferred.

Owing to the pharmacological properties, the abovementioned compounds are suitable for the prophylaxis and therapy of acute or chronic diseases which are caused by elevated levels of von Willebrand factor in the blood and/or increased expression of P-selectin.

These include thrombotic disorders provoked by ischemic states with subsequent reperfusion; such as thromboses in acute myocardial, mesenteric or else cerebral infarction; thrombotic disorders occurring during or after surgical operations; pulmonary embolisms; deep vein thromboses as occur at an increased rate after prolonged restriction of blood flow, especially in the lower extremities, for example after prolonged lying or sitting, and imflammatory disorders as occur during ischemia and subsequent reperfusion, during vasculitis (e.g., associated with autoimmune disease or connective tissue disease).

These also include disorders which are caused by increased expression of P-selectin, such as incipient inflammatory reactions; but also prophylaxis and treatment of arteriosclerosis; and prophylaxis and treatment of cancer; also inflammation of joints and arthritic disorders such as rheumatoid arthritis.

Administration of the medicaments of the invention can take place by oral, inhalational, rectal or transdermal administration or by subcutaneous, intraarticular, intraperitoneal or intravenous injection. Oral administration is preferred.

The invention also relates to a pharmaceutical composition useful according to the invention, which, comprises a sodium-dependent chloride/bicarbonate exchanger inhibitor with a pharmaceutically suitable and physiologically tolerated carrier and, where appropriate, other suitable active ingredients, additives or excipients into a suitable dosage form.

The abovementioned sodium-dependent chloride/bicarbonate exchanger inhibitors are mixed with the additives suitable for this purpose, such as carriers, stabilizers or inert diluents, and converted by conventional methods into suitable dosage forms such as tablets, coated tablets, two-piece capsules, aqueous, alcoholic or oily suspensions or aqueous or oily solutions. Examples of inert carriers that can be used are gum arabic, magnesia, magnesium carbonate, potassium phosphate, lactose, glucose or starch, especially corn starch. Preparation can moreover take place both as dry and as wet granules. Examples of suitable oily carriers or solvents are vegetable or animal oils, such as sunflower oil or fish liver oil.

For subcutaneous, intraperitoneal or intravenous administration, the sodium-dependent chloride/bicarbonate exchanger inhibitors are converted into solution, suspension or emulsion if desired with the substances suitable for this purpose, such as solubilizers, emulsifiers or other excipients. Examples of suitable solvents are physiological saline or alcohols, e.g., ethanol, propanol, glycerol, as well as sugar solutions such as glucose or mannitol solutions, or else a mixture of the various solvents mentioned.

Also used are conventional aids such as carriers, disintegrants, binders, coating agents, swelling agents, glidants or lubricants, flavorings, sweeteners and solubilizers. Excipients which are frequently used and which may be mentioned are magnesium carbonate, titanium dioxide, lactose, mannitol and other sugars, talc, milk protein, gelatin, starch, cellulose and derivatives thereof, animal and vegetable oils such as fish liver oil, sunflower, peanut or sesame oil, polyethylene glycol and solvents such as, for example, sterile water and monohydric and polyhydric alcohols such as glycerol.

The sodium-dependent chloride/bicarbonate exchanger inhibitors are preferably produced and administered as pharmaceutical products in dosage units, where one unit contains as active ingredient a defined dose of the abovementioned compounds. They can for this purpose be administered orally in doses of from 0.01 mg/kg/day to 25.0 mg/kg/day, preferably 0.01 mg/kg/day to 5.0 mg/kg/day or parenterally in doses of from 0.001 mg/kg/day to 5 mg/kg/day, preferably 0.001 mg/kg/day to 2.5 mg/kg/day. The dosage may also be increased in severe cases. However, lower doses also suffice in many cases. These data relate to an adult weighing about 75 kg.

The sodium-dependent chloride/bicarbonate exchanger inhibitors can be employed alone or in combination with anticoagulant, platelet aggregation-inhibiting or fibrinolytic agents. Coadministration can take place, for example, with factor Xa inhibitors, standard heparin, low molecular weight heparins such as enoxaparin, dalteparin, certroparin, parnaparin or tinzaparin, direct thrombin inhibitors such as hirudin, aspirin, fibrinogen receptor antagonists, streptokinase, urokinase and/or tissue plasminogen activator (tPA).

In contrast to the previously described effects of inhibitors of the sodium-dependent chloride/bicarbonate exchanger on the aggregation of blood platelets, the abovementioned compounds also show inhibition of excessive release of von Willebrand factor. This novel antithrombotic action principle differs from the previously disclosed antithrombotic action principles in a crucial and advantageous manner in that

a) it acts only in ischemic tissue in the subsequent reperfusion phase, whereas other cells not affected by the ischemia (preischemic) will remain completely unaffected, and

b) there is no need to worry about any of the dangerous hemorrhagic complications during the lysis therapy.

### EXAMPLES

The invention is explained in more detail by means of examples below.

The following examples demonstrated the effects of an extracellular acidosis (pHₑₓ=6.4) and are specific inhibitors of the sodium-dependent chloride/bicarbonate exchanger (NCBE) on the intracellular pH (pHᵢ) and the release of von-Willebrand factor (vWF). All the examples were carried out with human umbilical vein endothelial cells (HUVEC). These comprise primary cell cultures isolated from the umbilical vein.

For the following examples, the cells were cultivated either on gelatinized glass plates (measurement of the intracellular proton concentration) or on cell culture plates (12-well culture plates, Falcon, N.J., USA; measurement of vWF release) after the first passage.

### Example 1

Measurement of the Intracellular pH

To measure the intracellular proton concentration (pHᵢ), the HUVECs were loaded with the pH-sensitive fluorescent dye BCECF-AM (2′,7′-bis(carboxyethyl)-5(6)-carboxyfluorescein). A Deltascan spectrofluorometer (PTI, Hamburg) was employed for the subsequent fluorescence measurement. This measuring system consists essentially of a UV light source, a monochromator, a photon detector and the Felix and Oscar software packages (PTI, Hamburg) for controlling the system via a computer. After alternate excitation with the wavelengths 439.5 nm (pH-independent) and 490 nm (pH-sensitive), the ratio of the measured emissions of the BCECF (ratio) was reported and the pH was found after a calibration. The measuring cell is designed so that the parameters of temperature and carbon dioxide partial pressure in the system are controlled during continuous perfusion. For the reperfusion simulation, the experimental conditions were set at 37° C. and a carbon dioxide partial pressure of 5% or 10% by gassing the system and perfusate. In the experiment there was initially preincubation with sodium bicarbonate buffer pHₑₓ 6.4 for 60 minutes in order to simulate respiratory metabolic acidosis. The initiation perfusion was then changed to sodium bicarbonate buffer of pH 7.4 with 10 μM histamine as reperfusion simulation.

These control experiments were compared with an experiment in which the NCBE inhibitor 4′-[5-formyl-4-(2-methoxyethoxy)-2-phenyl-1-imidazolylmethyl]-3′-methylsulfonylbiphenyl-2-sulfonylcyanamide (called compound 1 hereinafter) and an NHE inhibitor were added, each in a concentration of 10 μM to the reperfusion buffer.

The results of several experiments have been summarized in Tables 1 and 2.

Table 1: Intracellular pH during extracellular acidosis (pHᵢ (acidosis)) of at least 15 minutes and under control conditions (Co).

TABLE 1

| | |
|---|---|
| pHᵢ (Acidosis) | 6.53 ± 0.02 (mean ± SEM) |
| pHᵢ (Co) | 7.23 ± 0.02 (mean ± SEM) |

SEM is the standard deviation from the mean

Extracellular acidosis leads to intracellular acidification which persists during the acidosis. The intracellular acidotic pH is virtually identical to the extracellular pH (applied extracellular acidosis pHₑₓ=6.4).

Table 2: Reperfusion with experimental buffer containing the abovementioned compound 1 and cariporide, cariporide-containing control buffer (NHE) and control buffer (Co). The time to the half-maximum pHᵢ change after 60 minutes of acidosis was found from the measurements during the first 30 seconds after reperfusion.

TABLE 2

| | Time to Δ pH_{i, max}/2 [s] |
|---|---|
| | Mean ± SEM |
| Co | 18 ± 1.5 |
| NHE | 190 ± 2.9 |
| Compound 1 + | 450 ± 32.7 |
| cariporide | |

### Example 2

Measurement of vWF Release After Reperfusion

The measurements were carried out in a Heraeus Heracell incubator. This made it possible to calculate the umbilical vein endothelial cells under controlled physiological conditions (temperature 37° C., relative humidity 100%, pCO₂ constant at 5%) and to ensure rapid change of different cell culture media.

Said cells were initially incubated with acidotic medium (pH 6.4 composed of the ingredients: medium M199 w/Earle's & amino acids, w/L-glutamine, w/o NaHCO₃, w/o Hepes+0.084 g NaHCO₃/I) or pH standard medium (pH 7.4 composed of the ingredients: medium M199 w/Earle's & amino acids, w/L-glutamine, w/o NaHCO₃, w/o Hepes+2.200 g NaHCO₃/I) for one, three or 48 hours. Before starting the reperfusion, samples of supernatant were taken to determine the vWF concentration under acidotic conditions (vWF_{acidosis}) and control conditions (vWF_{co}). To simulate reperfusion, the medium was changed to one with a pH of 7.4 (ingredients: medium M199 w/Earle's & amino acids, w/L-glutamine, w/o NaHCO₃, w/o Hepes+2.200 g NaHCO₃/I+10 μM histamine) to which the abovementioned NCBE inhibitor compound 1 was added in a concentration of 10 μM. Change to the same medium without corresponding addition of inhibitor served as control.

The samples taken from the supernatant were used to determine the vWF concentration. This was done by an ELISA method (enzyme-linked immuno sorbent assay) using specific antibodies. The vWF content of standard human plasma (Behring, Marburg) is calculated using an international standard (2^{nd} International Standard 87/718; National Institute for Biological Standards and Control, London).

Table 3: vWF concentration in the cell supernatant under acidotic (vWF_{acidosis}) and under control conditions (vWF_{co}), measured after incubation for 15 minutes. The vWF concentration under control conditions is set at 100%.

TABLE 3

| | |
|---|---|
| vWF_{co} | 100% |
| vWF_{acdosis} (constitutive) | 46 ± 1.1% |
| vWF_{acidosis} (stimulated, histamine 50 μM) | 52 ± 2.5% |

The acidosis led to a distinct decrease in vWF secretion, both the constitutive secretion and the stimulated Weibel-Palade body secretion. The vWF secretion was reduced by a factor of 2 compared with control cells during acidosis (pHₑₓ=6.4).

Table 4: vWF secretion was measured during a 10-minute reperfusion time with stimulation. The vWF secretion of the control cells (vWF_{co}) was set at 100%. The vWF concentration during the reperfusion of preacidotic cells (vWF_{acidosis}) and the vWF concentration during reperfusion of preacidotic cells in the presence of 10 μM of the abovementioned compound 1 (vWF_{co}) have been indicated as values relative to the control values.

TABELLE 4

| | |
|---|---|
| vWF_{co} | 100% |
| vWF_{acidosis} | 193 ± 8.0% |
| vWF_{c1} | 157 ± 18% |

During the reperfusion there was a large increase in vWF secretion by a factor of 2. Blockade of the NCBE transporter with the abovementioned compound 1 reduces the increased vWF secretion by almost 50% and thus approaches the control values.

The examples showed that extracellular acidosis as present for example during ischemia led to an intracellular acidosis, resulting in reduced (constitutive and stimulated) vWF secretions. The subsequent reperfusion and stimulation of the endothelial cells brought about rapid intracellular realkalinization. There was a simultaneous enhancement of the increased vWF secretion. A delay of the realkalinization with the abovementioned compound 1 reduces the increased vWF secretion and thus the possible thrombosis. The data show that the intracellular pH is determined by the extracellular pH. Secretion by the endothelial cells is in turn determined by the intracellular pH. It is thus possible, by inhibiting realkalinization, to reduce greatly the known endothelial cell activation during the reperfusion phase and the worry, connected therewith, about rethrombosis (vWF secretion) and inflammation.

We claim:

1. A method for the prophylactic or therapeutic treatment of a patient that would be subject to an acute or chronic disease caused by elevated levels of von Willebrand factors in the blood or increased expression of P-selectin, comprising administering a therapeutically effective amount of a cellular sodium-dependent chloride/bicarbonate exchanger inhibitor to the patient.

2. The method according to claim 1, wherein the cellular sodium-dependent chloride/bicarbonate exchanger inhibitor is a compound of the formula I 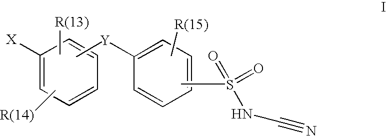
or a stereoisomeric form thereof or a mixture of stereoisomeric forms in any ratio, or a physiologically tolerated salt thereof, wherein: 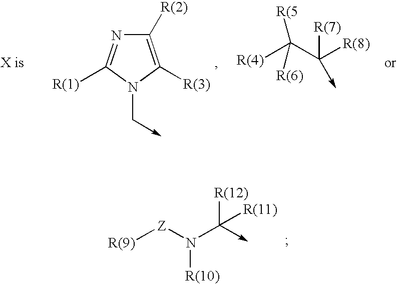
R(1) is hydrogen, alkyl having 1, 2, 3, 4, 5, 6, 7 or 8 carbon atoms, —CₐH₂ₐ-phenyl, which phenyl is unsubstituted or substituted by 1, 2 or 3 identical or different substituents selected from F, Cl, Br, I, CF₃, methyl, methoxy, hydroxyl and —NR(19)R(20), —C_{b}H_{2b}-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(21)R(22), or —C_{c}H_{2c}-cycloalkyl, which cycloalkyl has 3, 4, 5, 6 or 7 carbon atoms;
R(2) and R(3) are independently hydrogen, F, Cl, Br, I, CF₃, —CN, —NO₂, —CH₂OR(23), —CO—R(24), —O—R(25), —O-(alkylenyl having 2, 3, or 4 carbon atoms)-O—R(23), —NR(92)R(93), alkyl having 1, 2, 3, 4, 5, 6, 7 or 8 carbon atoms, cycloalkyl having 3, 4, 5, 6 or 7 carbon atoms, —C_{d}H_{2d}-phenyl, which phenyl is unsubstituted or substituted by 1, 2 or 3 identical or different substituents selected from F, Cl, Br, I, CF₃, methyl, methoxy, hydroxyl and —NR(33)R(34), —CₑH₂ₑ-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(35)R(36), or —SO_{f}—R(37);
R(4) is hydrogen, alkyl having 1, 2, 3, 4, 5, 6, 7 or 8 carbon atoms, 1-naphthyl, 2-naphthyl, —CᵢH₂ᵢ-cycloalkyl, which cycloalkyl has 3, 4, 5, 6 or 7 carbon atoms, or —CᵢH₂ᵢ-phenyl, which phenyl moiety is unsubstituted or substituted by 1, 2 or 3 identical or different substituents selected from alkyl having 1, 2, 3, 4, 5, 6, 7 or 8 carbon atoms, F, Cl, Br, I, CF₃, —SOⱼR(48), —OR(49), —NR(50)R(51), —CN, —NO₂ or —CO—R(52), or R(4) and R(6) taken together with the carbon atom bearing them form cycloalkyl, which cycloalkyl has 3, 4, 5, 6 or 7 carbon atoms, or fluorenyl;
R(5), R(6), R(7) and R(8) are independently hydrogen, F, CF₃, —O—R(56), alkyl having 1, 2, 3, 4, 5, 6, 7 or 8 carbon atoms, cycloalkyl having 3, 4, 5, 6 or 7 carbon atoms, —CₖH₂ₖ-phenyl, which the phenyl moiety is unsubstituted or substituted by 1, 2 or 3 identical or different substituents selected from F, Cl, Br, I, CF₃, methyl, methoxy, hydroxyl and —NR(57)R(58), or R(5) and R(7) taken together form a second bond between the carbon atoms bearing them;
R(9) is alkyl having 1, 2, 3, 4, 5, 6, 7 or 8 carbon atoms, alkenyl having 2, 3, 4, 5, 6, 7 or 8 carbon atoms, or -Q(1)-A;
R(10) is hydrogen, alkyl having 1, 2, 3, 4, 5, 6, 7 or 8 carbon atoms; alkenyl having 2, 3, 4, 5, 6, 7 or 8 carbon atoms, or -Q(2)—B,
R(11) and R(12) are independently hydrogen or alkyl having 1, 2, 3, 4, 5, 6, 7 or 8 carbon atoms;
R(13), R(14) and R(15) are independently hydrogen, alkyl having 1, 2, 3, 4, 5, 6, 7 or 8 carbon atoms, F, Cl, Br, I, CF₃, —CN, —NO₂, —SO_{q}—R(79), —CO—R(80), —O—R(81) or —O-(alkylenyl having 2, 3 or 4 carbon atoms)-O—R(95);
R(16) is hydrogen or —OR(85);
R(17) is hydrogen or alkyl having 1, 2, 3, 4, 5, 6, 7 or 8 carbon atoms;
R(18) is hydrogen, alkyl having 1, 2, 3, 4, 5, 6, 7 or 8 carbon atoms, —CO—R(87) or —SO₂R(88);
R(19) and R(20) are independently hydrogen or alkyl having 1, 2, 3 or 4 carbon atoms;
R(21) and R(22) are independently hydrogen or alkyl having 1, 2, 3 or 4 carbon atoms;
R(23) is hydrogen or alkyl having 1, 2, 3, 4, 5, 6, 7 or 8 carbon atoms;
R(23) is hydrogen or alkyl having 1, 2, 3, 4, 5, 6, 7 or 8 carbon atoms;
R(24) is hydrogen, alkyl having 1, 2, 3, 4, 5, 6, 7 or 8 carbon atoms, —OR(26), or phenyl, which phenyl is unsubstituted or substituted by 1, 2 or 3 identical or different substituents selected from F, Cl, Br, I, CF₃, methyl, methoxy, hydroxyl and —NR(27)R(28);
R(25) is hydrogen, alkyl having 1, 2, 3, 4, 5, 6, 7 or 8 carbon atoms, phenyl, which phenyl is unsubstituted or substituted by 1, 2 or 3 identical or different substituents selected from F, Cl, Br, I, CF₃, methyl, methoxy, hydroxyl and —NR(29)R(30), or heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(31)R(32);
R(26) is hydrogen or alkyl having 1, 2, 3, 4, 5, 6, 7 or 8 carbon atoms;
R(27) and R(28) are independently hydrogen or alkyl having 1, 2, 3 or 4 carbon atoms;
R(29) and R(30) are independently hydrogen or alkyl having 1, 2, 3 or 4 carbon atoms;
R(31) and R(32) are independently hydrogen or alkyl having 1, 2, 3 or 4 carbon atoms;
R(33) and R(34) are independently hydrogen or alkyl having 1, 2, 3 or 4 carbon atoms;
R(35) and R(36) are independently hydrogen or alkyl having 1, 2, 3 or 4 carbon atoms;
R(37) is alkyl having 1, 2, 3, 4, 5, 6, 7 or 8 carbon atoms, cycloalkyl having 3, 4, 5, 6 or 7 carbon atoms, or —C_{g}H_{2g}-phenyl, which phenyl is unsubstituted or substituted by 1, 2 or 3 identical or different substituents selected from F, Cl, Br, I, CF₃, methyl, methoxy, hydroxyl and —NR(38)R(39);
R(38) and R(39) are independently hydrogen or alkyl having 1, 2, 3 or 4 carbon atoms;
R(48) is alkyl having 1, 2, 3 or 4 carbon atoms or —NR(53)R(54);
R(49) is hydrogen or alkyl having 1, 2, 3 or 4 carbon atoms;
R(50) and R(51) are independently hydrogen or alkyl having 1, 2, 3 or 4 carbon atoms;
R(52) is hydrogen, alkyl having 1, 2, 3, 4, 5, 6, 7 or 8 carbon atoms or —OR(55);
R(53) and R(54) are independently hydrogen or alkyl having 1, 2, 3 or 4 carbon atoms;
R(55) is hydrogen, alkyl having 1, 2, 3, 4, 5, 6, 7 or 8 carbon atoms;
R(56) is hydrogen, alkyl having 1, 2, 3, 4, 5, 6, 7 or 8 carbon atoms, or phenyl which is unsubstituted or substituted by 1, 2 or 3 identical or different substituents selected from F, Cl, Br, I, CF₃, methyl, methoxy, hydroxyl and —NR(59)R(60), or heteroaryl having 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(61)R(62);
R(57) and R(58) are independently hydrogen or alkyl having 1, 2, 3 or 4 carbon atoms;
R(59) and R(60) are independently hydrogen or alkyl having 1, 2, 3 or 4 carbon atoms;
R(61) and R(62) are independently hydrogen or alkyl having 1, 2, 3 or 4 carbon atoms;
R(70) and R(71) are independently hydrogen, alkyl having 1, 2, 3, 4, 5, 6, 7 or 8 carbon atoms, -Q(3)-phenyl, which is unsubstituted or substituted by 1, 2 or 3 identical or different substituents selected from alkyl having 1, 2, 3, 4, 5, 6, 7 or 8 carbon atoms, F, Cl, Br, I, CF₃, —SOₚR(72), —OR(73), —NR(74)R(75), —CN, —NO₂, —CO—R(76), and alkenyl having 2, 3, 4, 5, 6, 7 or 8 carbon atoms;
R(63) and R(72) are independently alkyl having 1, 2, 3 or 4 carbon atoms or —NR(77)R(78);
R(64), R(65), R(66), R(68), R(69), R(73), R(74), R(75), R(77) and R(78) are independently hydrogen or alkyl having 1, 2, 3 or 4 carbon atoms;
R(67) and R(76) are independently hydrogen, alkyl having 1, 2, 3, 4, 5, 6, 7 or 8 carbon atoms or —OR(89);
R(79) is alkyl having 1, 2, 3, 4, 5, 6, 7 or 8 carbon atoms, —NR(82)R(83) or phenyl, which is unsubstituted or substituted by 1, 2 or 3 identical or different substituents selected from F, Cl, Br, I, CF₃, methyl, methoxy, hydroxyl and —NR(82)R(83);
R(80) is hydrogen, alkyl having 1, 2, 3, 4, 5, 6, 7 or 8 carbon atoms or —OR(84);
R(81) is hydrogen, alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl and —NR(82)R(83);
R(82) and R(83) are independently hydrogen or alkyl having 1, 2, 3 or 4 carbon atoms;
R(84) is hydrogen or alkyl having 1, 2, 3, 4, 5, 6, 7 or 8 carbon atoms;
R(85) is hydrogen, alkyl having 1, 2, 3, 4, 5, 6, 7 or 8 carbon atoms or —CO—R(86);
R(86) is alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, and hydroxyl;
R(87) and R(88) are independently alkyl having 1, 2, 3, 4, 5, 6, 7 or 8 carbon atoms, cycloalkyl having 3, 4, 5, 6, 7 or 8 carbon atoms or phenyl, which is unsubstituted or substituted by 1, 2 or 3 identical or different substituents selected from F, Cl, Br, I, CF₃, methyl, methoxy or hydroxyl;
R(89) is hydrogen or alkyl having 1, 2, 3, 4, 5, 6, 7 or 8 carbon atoms;
R(92) and R(93) independently of one another -(alkylenyl having 2, 3, or 4 carbon atoms)-O—R(94);
R(94) is hydrogen or alkyl having 1, 2, 3, 4, 5, 6, 7 or 8 carbon atoms;
R(95) is hydrogen or alkyl having 1, 2, 3, 4, 5, 6, 7 or 8 carbon atoms;
Z is carbonyl or sulfonyl;
A and B are independently aryl, which has 6, 7, 8, 9, 10, 11, 12, 13 or 14 carbon atoms, preferably phenyl, 1-naphthyl or 2-naphthyl, and is unsubstituted or substituted by 1, 2 or 3 identical or different substituents selected from alkyl having 1, 2, 3, 4, 5, 6, 7 or 8 carbon atoms, F, Cl, Br, I, CF₃, —SOₙR(63), —OR(64), —NR(65)R(66), —CN, —NO₂ and —CO—R(67), heteroaryl having 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(68)R(69), cycloalkyl having 3, 4, 5, 6 or 7 carbon atoms, —O—R(70), or —O—R(71);
Y is a covalent bond, CR(16)R(17), CO, S, SO₂, O or NR(18);
a is zero, 1 or 2
b is zero, 1 or 2;
c is zero, 1 or 2;
d is zero, 1 or 2;
e is zero, 1 or 2;
f is zero, 1 or 2;
g is zero, 1 or 2;
i is zero, 1 or 2;
j is zero, 1 or 2;
k is zero, 1 or 2;
n is zero, 1 or 2;
p is are independently zero, 1 or 2;
q are independently zero, 1, or 2;
Q(1) is a covalent bond, alkylenyl having 1, 2, 3 or 4 carbon atoms, or alkenylenyl having 2, 3 or 4 carbon atoms;
Q(2) is a covalent bond, alkylenyl having 1, 2, 3 or 4 carbon atoms, or alkenylenyl having 2, 3 or 4 carbon atoms; and
Q(3) is a covalent bond, alkylenyl having 1, 2, 3 or 4 carbon atoms, or alkenylenyl having 2, 3 or 4 carbon atoms.

3. The method according to claim 2, wherein the cellular sodium-dependent chloride/bicarbonate exchanger inhibitor is a compound of the formula I wherein X is 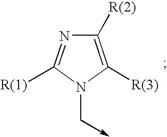
R(1) is alkyl having 1, 2, 3 or 4 carbon atoms or phenyl, which is unsubstituted or substituted by a substituent selected from F, Cl, CF₃, methyl or methoxy;
R(2) and R(3) are independently hydrogen, F, Cl, CF₃, —CN, CO—R(24), —O—R(25), alkyl having 1, 2, 3 or 4 carbon atoms, cycloalkyl having 3, 4, 5, 6 or 7 carbon atoms; or phenyl, which is unsubstituted or substituted by a substituent selected from F, Cl, CF₃, methyl or methoxy; heteroaryl having 1, 2, 3, 4, 5, 6, 7, 8 or 9 carbon atoms and is unsubstituted or substituted by a substituent selected from F, Cl, CF₃, methyl and methoxy, or —SO_{f}—R(37);
R(13), R(14) and R(15) are independently hydrogen, methyl, F, Cl, CF₃, —CN, —SO₂—R(79), —CO—R(80) or —O—R(81); and
R(24) is hydrogen, alkyl having 1, 2, 3 or 4 carbon atoms, —OR(26) or phenyl, which is unsubstituted or substituted by a substituent selected from F, Cl, CF₃, methyl and methoxy;
R(25) is hydrogen, alkyl having 1, 2, 3 or 4 carbon atoms, phenyl, which is unsubstituted or substituted by a substituent selected from F, Cl, CF₃, methyl and methoxy, or heteroaryl which is unsubstituted or substituted by a substituent selected from F, Cl, CF₃, methyl and methoxy;
R(26) is hydrogen, methyl or ethyl;
R(37) is alkyl having 1, 2, 3 or 4 carbon atoms or phenyl, which is unsubstituted or substituted by a substituent selected from F, Cl, CF₃, methyl and methoxy;
R(79) and R(81) are independently alkyl having 1, 2, 3 or 4 carbon atoms or phenyl, which is unsubstituted or substituted by a substituent selected from F, Cl, CF₃, methyl and methoxy;
R(80) is hydrogen, methyl or —OR(84);
R(84) is hydrogen or alkyl having 1, 2, 3, or 4 carbon atoms;
Y is a covalent bond or methylene; and
f is zero or 2.

4. The method according to claim 1, wherein the cellular sodium-dependent chloride/bicarbonate exchanger inhibitor is a compound of the formula II 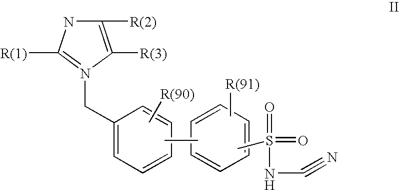
or a stereoisomeric form thereof or a mixture of stereoisomeric forms in any ratio, or a physiologically tolerated salt thereof,
wherein:
R(1) is hydrogen, alkyl having 1, 2, 3, 4, 5, 6, 7 or 8 carbon atoms, —CₐH₂ₐ-phenyl, which phenyl is unsubstituted or substituted by 1, 2 or 3 identical or different substituents selected from F, Cl, Br, I, CF₃, methyl, methoxy, hydroxyl and —NR(19)R(20), —C_{b}H_{2b}-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(21)R(22), or —C_{c}H_{2c}-cycloalkyl, which cycloalkyl has 3, 4, 5, 6 or 7 carbon atoms;
R(2) and R(3) are independently hydrogen, F, Cl, Br, I, CF₃, —CN, —NO₂, —CH₂OR(23), —CO—R(24), O—R(25), —O-(alkylenyl having 2, 3, or 4 carbon atoms)-O—R(23), or —NR(92)R(93), alkyl having 1, 2, 3, 4, 5, 6, 7 or 8 carbon atoms, cycloalkyl having 3, 4, 5, 6 or 7 carbon atoms or —C_{d}H_{2d}-phenyl, which phenyl is unsubstituted or substituted by 1, 2 or 3 identical or different substituents selected from F, Cl, Br, I, CF₃, methyl, methoxy, hydroxyl and —NR(33)R(34), —CₑH₂ₑ-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(35)R(36), or —SO_{f}—R(37);
R(19) and R(20) are independently hydrogen or alkyl having 1, 2, 3 or 4 carbon atoms;
R(21) and R(22) are independently hydrogen or alkyl having 1, 2, 3 or 4 carbon atoms;
R(23) is hydrogen or alkyl having 1, 2, 3, 4, 5, 6, 7 or 8 carbon atoms
R(24) is hydrogen, alkyl having 1, 2, 3, 4, 5, 6, 7 or 8 carbon atoms, —OR(26) or phenyl, which is unsubstituted or substituted by 1, 2 or 3 identical or different substituents selected from F, Cl, Br, I, CF₃, methyl, methoxy, hydroxyl and —NR(31)R(32);
R(25) is hydrogen, alkyl having 1, 2, 3, 4, 5, 6, 7 or 8 carbon atoms, phenyl, which is unsubstituted or substituted by 1, 2 or 3 identical or different substituents selected from F, Cl, Br, I, CF₃, methyl, methoxy, hydroxyl and —NR(29)R(30), or heteroaryl which has 1, 2, 3, 4, 5, 6, 7, 8 or 9 carbon atoms and is unsubstituted or substituted by 1, 2 or 3 identical or different substituents selected from F, Cl, Br, I, CF₃, CH₃, methoxy, hydroxyl and —NR(31)R(32);
R(26) is hydrogen or alkyl having 1, 2, 3, 4, 5, 6, 7 or 8 carbon atoms;
R(27) and R(28) are independently hydrogen or alkyl having 1, 2, 3 or 4 carbon atoms;
R(29) and R(30) are independently hydrogen or alkyl having 1, 2, 3 or 4 carbon atoms;
R(31) and R(32) are independently hydrogen or alkyl having 1, 2, 3 or 4 carbon atoms;
R(33) and R(34) are independently hydrogen or alkyl having 1, 2, 3 or 4 carbon atoms;
R(35) and R(36) are independently hydrogen or alkyl having 1, 2, 3 or 4 carbon atoms;
R(37) is alkyl having 1, 2, 3, 4, 5, 6, 7 or 8 carbon atoms, cycloalkyl having 3, 4, 5, 6 or 7 carbon atoms or —C_{g}H_{2g}-phenyl, which phenyl is unsubstituted or substituted by 1, 2 or 3 identical or different substituents selected from the group consisting of F, Cl, Br, I, CF₃, methyl, methoxy, hydroxyl and —NR(38)R(39);
R(38) and R(39) are independently hydrogen or alkyl having 1, 2, 3 or 4 carbon atoms;
R(79) is alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl and —NR(82)R(83);
R(80) is hydrogen, alkyl having 1, 2, 3, 4, 5, 6, 7 or 8 carbon atoms or —OR(84);
R(81) is hydrogen, alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl and —NR(82)R(83);
R(82) and R(83) are independently hydrogen or alkyl having 1, 2, 3 or 4 carbon atoms;
R(84) is hydrogen or alkyl having 1, 2, 3, 4, 5, 6, 7 or 8 carbon atoms;
R(90) and R(91) are independently hydrogen, alkyl having 1, 2, 3, 4, 5, 6, 7 or 8 carbon atoms, F, Cl, Br, I, CF₃, —CN, —NO₂, —SO_{q}—R(79), —CO—R(80), —O—R(81) or —O-(alkylenyl having 2, 3 or 4 carbon atoms)-O—R(95);
R(92) and R(93) are independently -(alkylenyl having 2, 3, or 4 carbon atoms)-O—R(94);
R(94) is hydrogen or alkyl having 1, 2, 3, 4, 5, 6, 7 or 8 carbon atoms;
R(95) is hydrogen or alkyl having 1, 2, 3, 4, 5, 6, 7 or 8 carbon atoms;
a is zero, 1 or 2;
b is zero, 1 or 2;
c is zero, 1 or 2;
d is zero, 1 or 2;
e is zero, 1 or 2;
f is zero, 1 or 2;
g is zero, 1 or 2; and
q is zero, 1 or 2.

5. The method according to claim 4, wherein the cellular sodium-dependent chloride/bicarbonate exchanger inhibitor is a compound of the formula II wherein
R(1) is hydrogen, alkyl having 1, 2, 3, 4, 5, 6, 7 or 8 carbon atoms, —CₐH₂ₐ-phenyl, which phenyl is unsubstituted or substituted by 1, 2 or 3 identical or different substituents selected from F, Cl, Br, I, CF₃, methyl, methoxy, hydroxyl and —NR(19)R(20), —C_{b}H_{2b}-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(21)R(22) or —C_{c}H_{2c}-cycloalkyl, which cycloalkyl has 3, 4, 5, 6 or 7 carbon atoms;
R(2) and R(3) are independently hydrogen, F, Cl, Br, I, CF₃, —CN, —NO₂, CH₂OR(23), CO—R(24), —O—R(25), alkyl having 1, 2, 3, 4, 5, 6, 7 or 8 carbon atoms, cycloalkyl having 3, 4, 5, 6 or 7 carbon atoms or —C_{d}H_{2d}-phenyl, which phenyl is unsubstituted or substituted by 1, 2 or 3 identical or different substituents selected from F, Cl, Br, I, CF₃, methyl, methoxy, hydroxyl and —NR(33)R(34),—CₑH₂ₑ-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(35)R(36), or —SO_{f}—R(37);
R(19) and R(20) are independently hydrogen or alkyl having 1, 2, 3 or 4 carbon atoms;
R(21) and R(22) are independently hydrogen or alkyl having 1, 2, 3 or 4 carbon atoms;
R(23) is hydrogen or alkyl having 1, 2, 3, 4, 5, 6, 7 or 8 carbon atoms;
R(24) is hydrogen, alkyl having 1, 2, 3, 4, 5, 6, 7 or 8 carbon atoms, —OR(26) or phenyl, which is unsubstituted or substituted by 1, 2 or 3 identical or different substituents selected from F, Cl, Br, I, CF₃, methyl, methoxy, hydroxyl and —NR(31)R(32);
R(25) is hydrogen, alkyl having 1, 2, 3, 4, 5, 6, 7 or 8 carbon atoms, phenyl, which is unsubstituted or substituted by 1, 2 or 3 identical or different substituents selected from F, Cl, Br, I, CF₃, methyl, methoxy, hydroxyl and —NR(29)R(30), or heteroaryl, which has 1, 2, 3, 4, 5, 6, 7, 8 or 9 carbon atoms and is unsubstituted or substituted by 1, 2 or 3 identical or different substituents selected from F, Cl, Br, I, CF₃, CH₃, methoxy, hydroxyl and —NR(31)R(32);
R(26) is hydrogen or alkyl having 1, 2, 3, 4, 5, 6, 7 or 8 carbon atoms;
R(29) and R(30) are independently hydrogen or alkyl having 1, 2, 3 or 4 carbon atoms;
R(31) and R(32) are independently hydrogen or alkyl having 1, 2, 3 or 4 carbon atoms;
R(33) and R(34) are independently hydrogen or alkyl having 1, 2, 3 or 4 carbon atoms;
R(35) and R(36) are independently hydrogen or alkyl having 1, 2, 3 or 4 carbon atoms;
R(37) is alkyl having 1, 2, 3, 4, 5, 6, 7 or 8 carbon atoms, cycloalkyl having 3, 4, 5, 6 or 7 carbon atoms or —C_{g}H_{2g}-phenyl, which phenyl is unsubstituted or substituted by 1, 2 or 3 identical or different substituents selected from the group consisting of F, Cl, Br, I, CF₃, methyl, methoxy, hydroxyl and —NR(38)R(39);
R(38) and R(39) are independently hydrogen or alkyl having 1, 2, 3 or 4 carbon atoms;
R(79) is alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl and NR(82)R(83);
R(82) and R(83) are independently hydrogen or alkyl having 1, 2, 3 or 4 carbon atoms;
R(91) is hydrogen,
R(90) is —SO_{q}—R(79),
a is zero, 1 or 2;
b is zero, 1 or 2;
c is zero, 1 or 2;
d is zero, 1 or 2;
e is zero, 1 or 2;
f is zero, 1 or 2;
g is zero, 1 or 2; and
q is zero, 1 or 2.

6. The method according to claim 4, wherein the cellular sodium-dependent chloride/bicarbonate exchanger inhibitor is a compound of the formula II wherein
R(1) is alkyl having 1, 2, 3, 4, 5, 6, 7 or 8 carbon atoms, —CₐH₂ₐ-phenyl, which phenyl is unsubstituted or substituted by 1, 2 or 3 identical or different substituents selected from F, Cl, Br, I, CF₃, methyl, methoxy, hydroxyl and —NR(19)R(20), or —C_{c}H_{2c}-cycloalkyl, which cycloalkyl has 3, 4, 5, 6 or 7 carbon atoms;
R(2) is hydrogen, F, Cl, Br, I, —O—R(25) or —SO_{f}—R(37);
R(3) is hydrogen, —CN, or CO—R(24);
R(19) and R(20) are independently hydrogen or alkyl having 1, 2, 3 or 4 carbon atoms;
R(24) is hydrogen, alkyl having 1, 2, 3, 4, 5, 6, 7 or 8 carbon atoms or OR(26);
R(25) is hydrogen, alkyl having 1, 2, 3, 4, 5, 6, 7 or 8 carbon atoms, or phenyl, which is unsubstituted or substituted by 1, 2 or 3 identical or different substituents selected from F, Cl, Br, I, CF₃, methyl, methoxy, hydroxyl or —NR(29)R(30);
R(26) is hydrogen or alkyl having 1, 2, 3, 4, 5, 6, 7 or 8 carbon atoms;
R(29) and R(30) are independently hydrogen or alkyl having 1, 2, 3 or 4 carbon atoms;
R(37) is alkyl having 1, 2, 3, 4, 5, 6, 7 or 8 carbon atoms, cycloalkyl having 3, 4, 5, 6 or 7 carbon atoms or —C_{g}H_{2g}-phenyl, which phenyl is unsubstituted or substituted by 1, 2 or 3 identical or different substituents selected from the group consisting of F, Cl, Br, I, CF₃, methyl, methoxy, hydroxyl and —NR(38)R(39);
R(38) and R(39) are independently hydrogen or alkyl having 1, 2, 3 or 4 carbon atoms;
R(79) is alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl and —NR(82)R(83); and
R(82) and R(83) are independently hydrogen or alkyl having 1, 2, 3 or 4 carbon atoms;
R(91) is hydrogen;
R(90) is —SO_{q}—R(79);
a is zero, 1 or 2;
c is zero, 1 or 2;
f is zero, 1 or 2;
g is zero, 1 or 2; and
q is zero, 1 or 2.

7. The method according to claim 4, wherein the cellular sodium-dependent chloride/bicarbonate exchanger inhibitor is a compound of the formula II wherein
R(1) is —CₐH₂ₐ-phenyl, which phenyl is unsubstituted or substituted by 1 or 2 identical or different substituents selected from F, Cl, Br, CF₃, methyl, methoxy, hydroxyl or —NR(19)R(20);
R(2) is F, Cl, Br, I or OR(25), in particular Cl;
R(3) is CO—R(24);
R(19) and R(20) are independently hydrogen or methyl;
R(24) and R(91) are hydrogen;
R(25) is alkyl having 1, 2, 3 or 4 carbon atoms;
R(79) equal to alkyl having 1, 2, 3 or 4 carbon atoms;
R(90) is SO₂R(79); and
a is zero, 1 or 2.

8. The method according to claim 4, wherein the compound of the formula II is a compound of the formula IIa or IIb

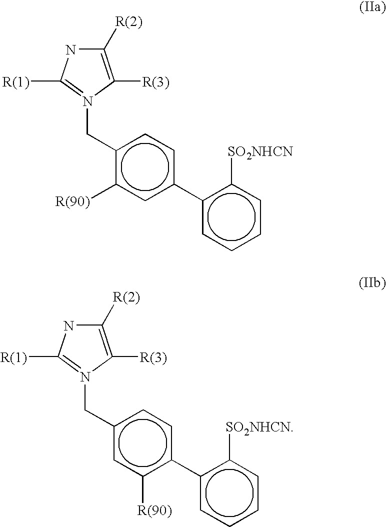

9. The method according to claim 4, wherein the cellular sodium-dependent chloride/bicarbonate exchanger inhibitor is a compound of the formula II wherein
R(1) is alkyl having 1, 2, 3, 4, 5, 6, 7 or 8 carbon atoms, —CₐH₂ₐ-phenyl, which phenyl is unsubstituted or substituted by 1, 2 or 3 identical or different substituents selected from F, Cl, Br, I, CF₃, methyl, methoxy, hydroxyl and —NR(19)R(20), —C_{b}H_{2b}-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl and —NR(21)R(22), or; or —C_{c}H_{2c}-cycloalkyl, which cycloalkyl has 3, 4, 5, 6 or 7 carbon atoms;
R(2) and R(3) are independently hydrogen, F, Cl, Br, I, CF₃, —CN, —NO₂, —CH₂OR(23), —CO—R(24), —O—R(25), —O-(alkylenyl having 2, 3, or 4 carbon atoms)-O—R(23), —NR(92)R(93), alkyl having 1, 2, 3, 4, 5, 6, 7 or 8 carbon atoms, cycloalkyl having 3, 4, 5, 6 or 7 carbon atoms, —C_{d}H_{2d}-phenyl, which phenyl is unsubstituted or substituted by 1, 2 or 3 identical or different substituents selected from F, Cl, Br, I, CF₃, methyl, methoxy, hydroxyl or —NR(33)R(34), —C_{c}H_{2c}-heteroaryl, which heteroaryl has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl or —NR(35)R(36), or —SO_{f}—R(37), and at least one of R(2) or R(3) is —O-(alkylenyl having 2, 3, or 4 carbon atoms)-O—R(23) or —NR(92)R(93);
R(19) and R(20) are independently hydrogen or alkyl having 1, 2, 3 or 4 carbon atoms;
R(21) and R(22) are independently hydrogen or alkyl having 1, 2, 3 or 4 carbon atoms;
R(23) is hydrogen or alkyl having 1, 2, 3, 4, 5, 6, 7 or 8 carbon atoms
R(24) is hydrogen, alkyl having 1, 2, 3, 4, 5, 6, 7 or 8 carbon atoms, —OR(26) or phenyl, which is unsubstituted or substituted by 1, 2 or 3 identical or different substituents selected from F, Cl, Br, I, CF₃, methyl, methoxy, hydroxyl and —NR(27)R(28);
R(25) hydrogen, alkyl having 1, 2, 3, 4, 5, 6, 7 or 8 carbon atoms, or phenyl, which is unsubstituted or substituted by 1, 2 or 3 identical or different substituents selected from F, Cl, Br, I, CF₃, methyl, methoxy, hydroxyl or —NR(29)R(30), or heteroaryl, which has 1, 2, 3, 4, 5, 6, 7, 8 or 9 carbon atoms and is unsubstituted or substituted by 1, 2 or 3 identical or different substituents selected from F, Cl, Br, I, CF₃, methyl, methoxy, hydroxyl or —NR(31)R(32);
R(26) is hydrogen or alkyl having 1, 2, 3, 4, 5, 6, 7 or 8 carbon atoms;
R(27) and R(28) are independently hydrogen or alkyl having 1, 2, 3 or 4 carbon atoms;
R(29) and R(30) are independently hydrogen or alkyl having 1, 2, 3 or 4 carbon atoms;
R(31) and R(32) are independently hydrogen or alkyl having 1, 2, 3 or 4 carbon atoms; or
R(33) and R(34) are independently hydrogen or alkyl having 1, 2, 3 or 4 carbon atoms;
R(35) and R(36) are independently hydrogen or alkyl having 1, 2, 3 or 4 carbon atoms;
R(37) is alkyl having 1, 2, 3, 4, 5, 6, 7 or 8 carbon atoms, cycloalkyl having 3, 4, 5, 6 or 7 carbon atoms or —C_{g}H_{2g}-phenyl which is unsubstituted or substituted by 1, 2 or 3 identical or different substituents selected from F, Cl, Br, I, CF₃, methyl, methoxy, hydroxyl or —NR(38)R(39);
R(38) and R(39) are independently hydrogen or alkyl having 1, 2, 3 or 4 carbon atoms;
R(79) alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl or —NR(82)R(83);
R(82) and R(83) are independently hydrogen or alkyl having 1, 2, 3, or 4 carbon atoms;
R(80) is hydrogen, alkyl having 1, 2, 3, 4, 5, 6, 7 or 8 carbon atoms or OR(84);
R(84) is hydrogen, alkyl having 1, 2, 3, 4, 5, 6, 7 or 8 carbon atoms;
R(81) is hydrogen, alkyl having 1, 2, 3, 4, 5, 6, 7 or 8 carbon atoms or phenyl, which is unsubstituted or substituted by 1, 2 or 3 identical or different substituents selected from F, Cl, Br, I, CF₃, methyl, methoxy, hydroxyl or —NR(82)R(83);
R(82) and R(83) are independently hydrogen or alkyl having 1, 2, 3 or 4 carbon atoms;
R(90) and R(91) are independently hydrogen, alkyl having 1, 2, 3, 4, 5, 6, 7 or 8 carbon atoms, F, Cl, Br, I, CF₃, —CN, —NO₂, —SO_{q}—R(79), —CO—R(80), —O—R(81) or —O-(alkylenyl having 2, 3 or 4 carbon atoms)-O—R(95);
R(92) and R(93) are independently -(alkylenyl having 2, 3, or 4 carbon atoms)-O—R(94);
R(94) is hydrogen or alkyl having 1, 2, 3, 4, 5, 6, 7 or 8 carbon atoms;
R(95) is hydrogen or alkyl having 1, 2, 3, 4, 5, 6, 7 or 8 carbon atoms;
a is zero, 1 or 2;
b is zero, 1 or 2;
c is zero, 1 or 2;
d is zero, 1 or 2;
f is zero, 1 or 2;
g is zero, 1 or 2; and
q is zero, 1 or 2.

10. The method according to claim 4, wherein the cellular sodium-dependent chloride/bicarbonate exchanger inhibitor is 4′-[5-formyl-4-(2-methoxyethoxy)-2-phenyl-1-imidazolylmethyl]-3′-methylsulfonylbiphenyl-2-sulfonylcyanamide or 4′-{[benzyl(thiophene-2-sulfonyl)amino]methyl}-3′-methanesulfonylbiphenyl-2-sulfonylcyanamide.

11. The method according to claim 1, wherein the cellular sodium-dependent chloride/bicarbonate exchanger inhibitor is a compound of the formula III 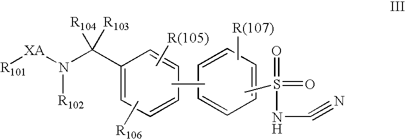
or a stereoisomeric form thereof or a mixture of stereoisomeric forms in any ratio, or a physiologically tolerated salt thereof,
wherein:
R(101) is alkyl, which has 1, 2, 3, 4, 5, 6, 7 or 8 carbon atoms and is optionally substituted by fluorine at one to all of the hydrogen atoms thereof, alkenyl having 2, 3, 4, 5, 6, 7, 8, 9, 10, 11 or 12 carbon atoms; -Q(4)—Y,
R(102) is hydrogen; alkyl, which has 1, 2, 3, 4, 5, 6, 7 or 8 carbon atoms and is optionally substituted by fluorine at one to all of the hydrogen atoms thereof, alkenyl having 2, 3, 4, 5, 6, 7, 8, 9, 10, 11 or 12 carbon atoms, alkynyl having 2, 3, 4, 5, 6, 7 or 8 carbon atoms, -Q(5)—Y,
R(103) and R(104) are independently hydrogen or alkyl having 1, 2, 3, 4, 5, 6, 7 or 8 carbon atoms;
R(105), R(106)and R(107) are independently hydrogen, alkyl having 1, 2, 3, 4, 5, 6, 7 or 8 carbon atoms, F, Cl, Br, I, CF₃, —CN, —NO₂, —SO_{q}—R(79), —CO—R(80), —O—R(81), or —O-(alkylenyl having 2, 3, or 4 carbon atoms)-O—R(95);
R(70) and R(71) are independently hydrogen, alkyl having 1, 2, 3, 4, 5, 6, 7 or 8 carbon atoms, -Q(3)-phenyl, which is unsubstituted or substituted by 1, 2 or 3 identical or different substituents selected from alkyl having 1, 2, 3, 4, 5, 6, 7 or 8 carbon atoms, F, Cl, Br, I, CF₃, —SOₚR(72), —OR(73), —NR(74)R(75), —CN, —NO₂, —CO—R(76), and alkenyl having 2, 3, 4, 5, 6, 7 8, 9, 10, 11 or 12 carbon atoms;
R(72) is alkyl, which has 1, 2, 3 or 4 carbon atoms and is optionally substituted by fluorine at one to all of the hydrogen atoms thereof, or —NR(77)R(78);
R(68), R(69), R(73), R(74), R(75) R(77) and R(78) are independently hydrogen or alkyl having 1, 2, 3 or 4 carbon atoms;
(76) is hydrogen, alkyl having 1, 2, 3, 4, 5, 6, 7 or 8 carbon atoms or —OR(89);
R(79) is alkyl having 1, 2, 3, 4, 5, 6, 7 or 8 carbon atoms, —NR(82)R(83) or phenyl which is unsubstituted or substituted by 1, 2 or 3 identical or different substituents selected from F, Cl, Br, I, CF₃, methyl, methoxy, hydroxyl and —NR(82)R(83);
R(80) is hydrogen, alkyl having 1, 2, 3, 4, 5, 6, 7 or 8 carbon atoms or OR(84);
R(81) hydrogen, alkyl having 1, 2, 3, 4, 5, 6, 7 or 8 carbon atoms, optionally substituted by (C₁-C₄)-alkoxy, or phenyl which is unsubstituted or substituted by 1, 2 or 3 identical or different substituents selected from F, Cl, Br, I, CF₃, methyl, methoxy, hydroxyl or NR(82)R(83);
R(82), R(83), R(108) and R(109) are independently hydrogen, alkyl having 1, 2, 3 or 4 carbon atoms or (C₁-C₄)-alkanoyl;
R(84) is hydrogen or alkyl having 1, 2, 3, 4, 5, 6, 7 or 8 carbon atoms;
R(89) is hydrogen or alkyl having 1, 2, 3, 4, 5, 6, 7 or 8 carbon atoms;
R(95) is hydrogen or alkyl having 1, 2, 3, 4, 5, 6, 7 or 8 carbon atoms;
R(96) and R(97) are independently hydrogen or CO—OR(99);
R(98) is hydrogen, alkyl, which has 1, 2, 3 or 4 carbon atoms, and is optionally substituted by fluorine at one to all of the hydrogen atoms thereof, preferably CF₃, or optionally substituted by alkoxy, aryl having 6, 7, 8, 9, 10, 11, 12, 13 or 14 carbon atoms, and is optionally substituted by 1, 2 or 3 identical or different substituents selected from F, Cl, Br, I, CF₃, NR(108)R(109), —CN, and —NO₂;
R(99) hydrogen, alkyl having 1, 2, 3, 4, 5, 6, 7 or 8 carbon atoms or —CᵣH₂ᵣ-phenyl;
n is zero, 1 or 2;
p is zero, 1 or 2;
q is zero, 1 or 2;
r is equal to 1, 2, 3 or 4;
XA is carbonyl, —CO—NH—, —CO—CO— or sulfonyl;
Y and Z are independently aryl, which has 6, 7, 8, 9, 10, 11, 12, 13 or 14 carbon atoms, preferably phenyl, 1-naphthyl or 2-naphthyl, which is unsubstituted or substituted by 1, 2, 3, 4 or 5 identical or different substituents selected from alkyl having 1, 2, 3, 4, 5, 6, 7 or 8 carbon atoms, aryl having 6, 7, 8, 9, 10, 11, 12, 13 or 14 carbon atoms, F, Cl, Br, I, CF₃, SOₙR(72), —OR(73), —NR(74)R(75), —CN, —NO₂ and —CO—R(76), or where two substituents together form a fused heterocyclyl substitute, heteroaryl, which has 1, 2, 3, 4, 5, 6, 7, 8 or 9 carbon atoms and is substituted by 1, 2 or 3 identical or different substituents selected from F, Cl, Br, I, CF₃, methyl, methoxy, hydroxyl and —NR(68)R(69), cycloalkyl, which has 3, 4, 5, 6, 7, 8, 9 or 10 carbon atoms, and is optionally substituted by aryl having 6, 7, 8, 9, 10, 11, 12, 13 or 14 carbon atoms, —O—R(70), —O—R(71), —SO₂—R(70), arylalkylcarbonyl, or heterocyclyl;
Q(3) is a covalent bond, alkylenyl having 1, 2, 3 or 4 carbon atoms, or alkenylenyl having 2, 3 or 4 carbon atoms;
Q(4) is a covalent bond, alkylenyl having 1, 2, 3 or 4 carbon atoms, or alkenylenyl having 2, 3 or 4 carbon atoms, and 1, 2 or 3 hydrogen atoms in Q(4) are optionally substituted independently by a substituent selected from aryl having 6, 7, 8, 9, 10, 11, 12, 13 or 14 carbon atoms, amino, —NR(96)R(97), alkoxycarbonyl, COOR(98), alkyl having 1, 2, 3 or 4 carbon atoms or (C₆-C₁₄)-aryl-(C₁-C₄)-alkylcarbonyl; and
Q(5) is a covalent bond, alkylenyl having 1, 2, 3 or 4 carbon atoms, or alkenylenyl having 2, 3 or 4 carbon atoms, and 1, 2 or 3 hydrogen atoms in Q(5) are optionally substituted independently by a substituent selected from aryl having 6, 7, 8, 9, 10, 11, 12, 13 or 14 carbon atoms, amino, —NR(96)R(97), —(C₁-C₄)-alkoxycarbonyl, —COOR(98), or alkyl having 1, 2, 3 or 4 carbon atoms.

12. The method according to claim 1, wherein the cellular sodium-dependent chloride/bicarbonate exchanger inhibitor is a compound of the formula IV 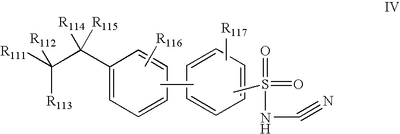
or a stereoisomeric form thereof or a mixture of stereoisomeric forms in any ratio, or a physiologically tolerated salt thereof, wherein:
R(111) is hydrogen, alkyl having 1, 2, 3, 4, 5, 6, 7 or 8 carbon atoms, 1-naphthyl, 2-naphthyl, —CᵢH₂ᵢ-cycloalkyl having 3, 4, 5, 6 or 7 carbon atoms or —CᵢH₂ᵢ-phenyl, where the phenyl moiety is unsubstituted or substituted by 1, 2 or 3 identical or different substituents selected from alkyl having 1, 2, 3, 4, 5, 6, 7 or 8 carbon atoms, F, Cl, Br, I, CF₃, SOⱼR(48), OR(49), NR(50)R(51), —CN, —NO₂ or CO—R(52), or R(111) and R(113) taken together with the carbon atom bearing them form cycloalkyl having 3, 4, 5, 6 or 7 carbon atoms or fluorenyl;
R(112), R(113), R(114) and R(115) are independently hydrogen, F, CF₃, O—R(56), alkyl having 1, 2, 3, 4, 5, 6, 7 or 8 carbon atoms, cycloalkyl having 3, 4, 5, 6 or 7 carbon atoms, —CₖH₂ₖ-phenyl, which phenyl is unsubstituted or substituted by 1, 2 or 3 identical or different substituents selected from the group F, Cl, Br, I, CF₃, methyl, methoxy, hydroxyl or NR(57)R(58);
R(112) and R(114) together a second bond between the carbon atoms bearing them;
R(116) and R(117) are independently hydrogen, alkyl having 1, 2, 3, 4, 5, 6, 7 or 8 carbon atoms, F, Cl, Br, I, CF₃, —CN, —NO₂, —SO_{q}—R(79), —CO—R(80), —O—R(81), or —O-(alkylenyl having 2, 3 or 4 carbon atoms)-O—R(95);
R(48) is alkyl having 1, 2, 3 or 4 carbon atoms or —NR(53)R(54);
R(49) hydrogen or alkyl having 1, 2, 3 or 4 carbon atoms;
R(50) and R(51) are independently hydrogen or alkyl having 1, 2, 3 or 4 carbon atoms;
R(52) is hydrogen, alkyl having 1, 2, 3, 4, 5, 6, 7 or 8 carbon atoms or OR(55);
R(53) and R(54) are independently hydrogen or alkyl having 1, 2, 3 or 4 carbon atoms;
R(55) is hydrogen, alkyl having 1, 2, 3, 4, 5, 6, 7 or 8 carbon atoms;
R(56) is hydrogen, alkyl having 1, 2, 3, 4, 5, 6, 7 or 8 carbon atoms, phenyl, which is unsubstituted or substituted by 1, 2 or 3 identical or different substituents selected from the group of F, Cl, Br, I, CF₃, methyl, methoxy, hydroxyl or NR(59)R(60), or heteroaryl, which has 1, 2, 3, 4, 5, 6, 7, 8 or 9-carbon atoms, and is unsubstituted or substituted by 1, 2 or 3 identical or different substituents selected from the group of F, Cl, Br, I, CF₃, methyl, methoxy, hydroxyl or NR(61)R(62);
R(57) and R(58) are independently hydrogen or alkyl having 1, 2, 3 or 4 carbon atoms;
R(59) and R(60) are independently hydrogen or alkyl having 1, 2, 3 or 4 carbon atoms;
R(61) and R(62) are independently hydrogen or alkyl having 1, 2, 3 or 4 carbon atoms;
R(79) is alkyl having 1, 2, 3, 4, 5, 6, 7 or 8 carbon atoms, —NR(82)R(83), phenyl, which is unsubstituted or substituted by 1, 2 or 3 identical or different substituents selected from the group of F, Cl, Br, I, CF₃, methyl, methoxy, hydroxyl or —NR(82)R(83);
R(80) is hydrogen, alkyl having 1, 2, 3, 4, 5, 6, 7 or 8 carbon atoms or OR(84);
R(84) and R(95) are indendently hydrogen, alkyl having 1, 2, 3, 4, 5, 6, 7 or 8 carbon atoms;
R(81) is hydrogen, alkyl having 1, 2, 3, 4, 5, 6, 7 or 8 carbon atoms or phenyl which is unsubstituted or substituted by 1, 2 or 3 identical or different substituents selected from the group of F, Cl, Br, I, CF₃, methyl, methoxy, hydroxyl or NR(82)R(83);
R(82) and R(83) are independently hydrogen or alkyl having 1, 2, 3 or 4 carbon atoms;
i is zero, 1 or 2;
j is zero, 1 or 2;
k is zero, 1 or 2; and
q is zero, 1 or 2.

13. The method according to claim 12, wherein the cellular sodium-dependent chloride/bicarbonate exchanger inhibitor is a compound of the formula IV wherein
R(111) is methyl, ethyl, 1-naphthyl, 2-naphthyl, cycloalkyl having 3, 4, 5, 6 or 7 carbon atoms or phenyl, which is unsubstituted or substituted by one substituent from the group of alkyl having 1, 2, 3 or 4 carbon atoms, F, Cl, CF₃, —SO₂R(48), —OR(49), —NR(50)R(51), or —CO—R(52), or R(111) and R(113) taken together with the carbon atom bearing them form cycloalkyl having 3, 4, 5, 6 or 7 carbon atoms or fluorenyl;
R(112) and R(114) are hydrogen, or taken together form a second bond between the carbon atoms bearing them;
R(113) and R(115) are independently hydrogen, CF₃, O—R(56), alkyl having 1, 2, 3 or 4 carbon atoms or phenyl which is unsubstituted or substituted by one substituent selected from F, Cl, CF₃, methyl, methoxy, hydroxyl or —NR(57)R(58);
R(116) and R(117) are independently hydrogen, F, Cl, CF₃, —SO₂-methyl, —CO—R(80) or —O—R(81);
R(48) methyl or dimethylamino;
R(49) hydrogen, methyl or ethyl;
R(50) and R(51) are independently hydrogen, methyl or ethyl;
R(52) hydrogen or alkyl having 1, 2, 3 or 4 carbon atoms;
R(57) and R(58) are independently hydrogen, methyl or ethyl;
R(56) is hydrogen, alkyl having 1, 2, 3 or 4 carbon atoms or phenyl which is unsubstituted or substituted by 1 substituent from the group of F, Cl, CF₃, methyl, methoxy, hydroxyl, NR(59)R(60) or heteroaryl having 1, 2, 3, 4, 5, 6, 7, 8 or 9 carbon atoms which is unsubstituted or substituted by one substituent selected from F, Cl, CF₃, methyl, methoxy, hydroxyl or dimethylamino;
R(59) and R(60) are independently hydrogen, methyl or ethyl;
R(80) hydrogen or alkyl having 1, 2, 3 or 4 carbon atoms;
R(81) hydrogen, alkyl having 1, 2, 3 or 4 carbon atoms or pheny, l which is unsubstituted or substituted by 1 substituent from the group of F, Cl, CF₃, methyl, methoxy, hydroxyl or —NR(82)R(83); and
R(82) and R(83) are independently hydrogen, methyl or ethyl.

14. The method according to claims 1 to 13 further comprising a sodium/hydrogen exchanger inhibitor.

15. The method according to claim 14, wherein the sodium/hydrogen exchanger inhibitor is a compound of the formula selected from 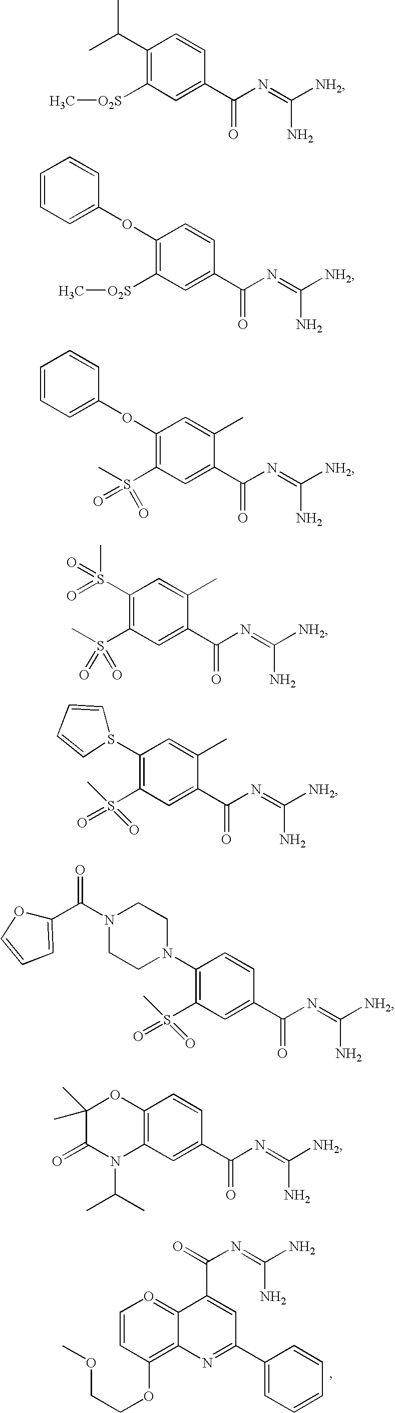 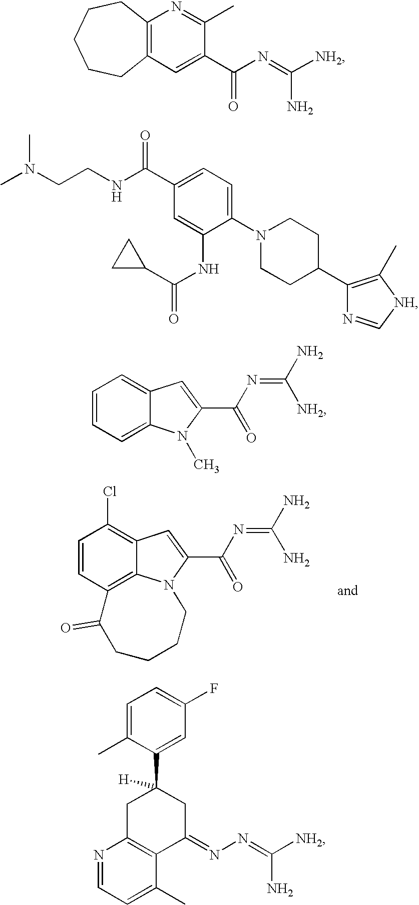
or a physiologically tolerated salt of the sodium/hydrogen exchanger inhibitor.

16. The method according to claim 15, wherein the sodium/hydrogen exchanger inhibitor is a compound of the formula

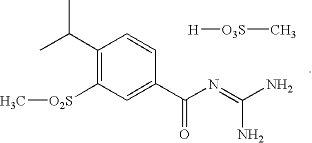

17. The method according to claim 16, wherein the cellular sodium-dependent chloride/bicarbonate exchanger inhibitor is 4′-[5-formyl-4-(2-methoxyethoxy)-2-phenyl-1-imidazolylmethyl]-3′-methylsulfonylbiphenyl-2-sulfonylcyanamide.

18. The method according to claim 17, wherein the disease is selected from a thrombotic disorder that is provoked by ischemic states with subsequent reperfusion, thrombotic disorder occurring during or after surgical operations, pulmonary embolism, deep vein thrombosis as occurs at an increased rate after prolonged restriction of blood flow, inflammatory disorders as occur during ischemia and subsequent reperfusion, vasculitis such as associated with an autoimmune disease or connective tissue disease, incipient inflammatory reaction, arteriosclerosis, cancer, or joint or arthritic inflammatory disorders.

19. The method according to claim 14, further comprising an anticoagulant, platelet aggregation-inhibitor or fibrinolytic agents.

20. The method according to claim 19, further comprising a factor Xa inhibitor, standard heparin, low molecular weight heparin, direct thrombin inhibitor, aspirin, fibrinogen receptor antagonist, streptokinase, urokinase or tissue plasminogen activator.

21. The method according to claim 1, wherein the treatment is effected by oral, inhalational, rectal or transdermal administration or by subcutaneous, intraarticular, intraperitoneal or intravenous injection.
